(19) United States
(12) Patent Application Publication
(10) Pub. No.: US 2023/0167423 A1
(43) Pub. Date: Jun. 1, 2023

(54) RING NUCLEASE

(71) Applicant: University Court of the University of St Andrews, Fife (GB)

(72) Inventors: Malcolm F. White, St. Andrews (GB); Januka Athukoralage, St. Andrews (GB)

(21) Appl. No.: 17/432,201

(22) PCT Filed: Feb. 19, 2020
(86) PCT No.: PCT/GB2020/050397

§ 371 Date: Aug. 19, 2021

(30) Foreign Application Priority Data
Feb. 19, 2019 (GB) 1902256.5

(51) Int. Cl. C12N 9/22; A61P 31/04; G01N 33/573

(52) U.S. Cl. CPC C12N 9/22; A61P 31/04; G01N 33/573; G01N 2500/02

(57) ABSTRACT

A family of structurally related proteins has been found to have enzymatic activity. The protein family may comprise DUF1874 proteins. Members of this family can be used to modulate the structure, function and/or activity of a cellular signalling molecule that is associated with a cellular antiviral response. In particular, the proteins described herein exhibit an ability to modulate the function, structure and/or activity of cyclic oligoadenylate (cOA); that is to say they can be used to inhibit, destroy, ablate and/or breakdown cOA activity, structure and/or function. The disclosed proteins (all of which belong to the DUF1874 protein family) are generally referred to as “ring nucleases”.

### RELATED APPLICATIONS

This application is a 35 U.S.C. § 371 national stage of PCT Application No. PCT/GB2020/050397, filed on Feb. 19, 2020, which claims priority from United Kingdom Patent Application No. 1902256.5, filed on Feb. 19, 2019, the contents of which are incorporated herein by reference. The above-referenced PCT International Application was published in the English language as International Publication No. WO 2020/169970 A1 on Aug. 27, 2020.

### STATEMENT REGARDING ELECTRONIC FILING OF A SEQUENCE LISTING

A Sequence Listing in ASCII text format, submitted under 37 C.F.R. § 1.821, entitled 9013.198 replacement seq list_ST25.txt, 34 kilobytes in size, generated on May 5, 2022, and filed via EFS-Web, is provided in lieu of a paper copy. This Sequence Listing is incorporated by reference into the specification for its disclosures.

### FIELD

The present invention is based on the identification of a family of enzymes which modulate the structure, activity and/or function of a signalling molecule associated with a cellular antiviral response.

### BACKGROUND

The rise of antimicrobial-resistant (AMR) pathogenic bacteria is a global health challenge, creating a need for novel treatments. Historically, viruses (known as “bacteriophages”) that naturally infect and kill bacteria were harnessed as a medical treatment (which is known as “phage therapy”). However, phage therapy was often found to yield inconsistent results.

More recently the discovery and characterisation of the CRISPR (clustered regularly interspaced short palindromic repeats) system has formed the basis of many technological developments, particularly focused around genome engineering. The potential to use the CRISPR system in medicine, in particular to develop more efficient phage therapies is also an area of interest. For example, one possibility may be to use a CRISPR system to knock-out cellular defenses, thus allowing a phage to kill the cell.

The use of some CRISPR systems in attacking and/or destroying antibiotic resistant bacterial pathogens has been reported (reviewed in Pursey et al (2018) CRISPR-Cas antimicrobials: Challenges and future prospects. PLoS Pathog 14(6):e1006990). Furthermore, a range of phage-encoded anti-CRISPR proteins, which are used by the phage to inhibit and overcome the cell's defenses, have been discovered (reviewed in Borges et al (2017) The Discovery, Mechanisms, and Evolutionary Impact of Anti-CRISPRs. Annu Rev Virol 4(1):37-59). These have found particular utility in the control of class 2 CRISPR systems (Cas9, Cas12) in genome engineering.

Type III CRISPR systems may be considered as one of the most potent cellular defenses. Type III CRISPR systems are known to synthesise the signalling molecule cyclic oligoadenylate (cOA) when they detect viral RNA (Kazlauskiene et al (2017) A cyclic oligonucleotide signaling pathway in type III CRISPR-Cas systems. Science 357(6351):605-609). cOA may be considered as a type of “alarm signal” that potentiates the antiviral response in cells, enabling them to destroy viral targets and/or halt infection.

Recently, a distinct cellular enzyme was identified that has the ability to breakdown or destroy cOA molecules. The destruction of cOA molecules in this way allows the cell to reset itself to an uninfected state (Athukoralage et al (2018) Ring nucleases deactivate Type III CRISPR ribonucleases by degrading cyclic oligoadenylate, Nature 562(7726):277-280). These cellular enzymes may be referred to herein as cellular ring nucleases (cRN).

### SUMMARY

The present invention is based on the identification of enzymatic activity within a family of structurally related proteins. The protein family may comprise DUF1874 proteins. Each member of this family can be used to modulate the structure, function and/or activity of a cellular signalling molecule. In particular, the proteins described herein exhibit an ability to modulate the function, structure and/or activity of cyclic oligoadenylate (cOA); that is to say they can be used to inhibit, destroy, ablate and/or breakdown cOA activity, structure and/or function. The disclosed proteins (all of which belong to the DUF1874 protein family) are generally referred to as “ring nucleases”.

The ring nucleases of this disclosure may be obtained or derived from viruses, including those viruses referred to as archaeal viruses and bacteriophages. Alternatively, ring nucleases embraced within the scope of this disclosure may be obtained or derived from bacteria and microorganisms known as archaea where ring nuclease sequences can reside as pro-viruses or prophages integrated within bacterial and/or archaeal genomes.

The ring nucleases of this disclosure are characterised by a high level of activity, broad specificity and (as described in more detail below) significant utility.

Upon detecting viral DNA, cellular type III CRISPR systems synthesise cOA (cyclic oligoadenylate); this acts as a “signal” potentiating an antiviral response in the cell. The role of the disclosed ring nuclease enzymes is to modulate (that is inhibit, destroy, ablate and/or breakdown the structure and/or function of) the cOA signalling molecule; this re-sets the cell to an uninfected state, neutralizes the type III CRISPR system and allows the virus to continue to replicate.

The term “cOA” embraces a class of cyclic molecules that are made up of a number of adenosine monophosphate units (AMP). Cyclic oligoadenylate molecules may be present in a range of ring sizes, typically comprising from 3 to 6 AMP subunits. These are denoted as cA3 (for a ring containing 3 AMP subunits), cA4 (for a ring containing 4 AMP subunits) and so on.

The cA4 signalling molecule may be present in many bacteria and examples of bacterial type III CRISPR systems which exploit cA4 or cA6 as the signalling molecule have been described. In some embodiments, ring nucleases have been found to degrade both cA4 and cA6 (e.g. cOA molecules with defined ring sizes of 4 and 6 AMP subunits respectively).

The ring nucleases of this disclosure may modulate cOA function, structure and/or activity by catalysing, facilitating and/or promoting the breakdown and/or degradation of one or more cOA molecules, e.g. one or more of cA3 to cA6. In particular, the disclosed ring nucleases may modulate (i.e. inhibit, breakdown, or degrade or alter) cA4 and/or cA6 function, structure and/or activity.

Modulation of cOA structure, function and/or activity by a ring nuclease may result in the destruction of the cOA cyclic structure and/or may produce one or more cOA fragments. The modulation (e.g. degradation and/or breakdown) of the cOA structure may be such that these cOA fragments are no longer able to perform the signalling function associated with initiating or potentiating an antiviral response. Additionally, the ring nucleases of this disclosure may share a central binding pocket lined by conserved residues important in catalysis. For example, the ring nucleases of this disclosure possess a highly conserved catalytic site that could facilitate the degradation of cOA molecules. In particular, it is hypothesised that the activity (e.g. the degradative ability) of these ring nucleases may involve and/or may be promoted by a histidine residue within this catalytic site.

Many anti-CRISPR (aCR) systems rely on specific protein:protein interactions and/or may function via a “spanner in the works” type mechanism (e.g. by blocking metabolic pathways in the cell or the like). Consequently such systems are generally constrained to have a high specificity for a particular target protein in one genus of bacteria. By contrast, and without wishing to be bound by theory, a single ring nuclease enzyme is likely to have a broad utility in the inhibition of endogenous type III CRISPR systems. Additionally, and again without wishing to be bound by theory, unlike prior art ring nucleases, the enzymes disclosed herein appear to modulate cOA function and/or activity via a different catalytic mechanism and/or a different cOA binding site. Indeed, crystal structure analysis of several of the sequences disclosed herein shows that the overall fold of the protein is very different from prior art enzymes.

In addition, the ring nucleases disclosed herein may modulate cOA structure, function and/or activity at temperatures which are typical growth temperatures for many bacteria. For example, typical bacterial growth temperatures may be in the range 20 to 40° C. or 25 to 37° C. By way of further example, the ring nucleases disclosed herein may have an optimum temperature range corresponding to or correlating with a typical bacterial growth temperature. Additionally or alternatively, the ring nucleases disclosed herein may function or work most effectively (e.g. in terms of activity, potency and/or specificity) at temperatures between 20 and 40° C., or between 25 and 37° C. In contrast, the prior art ring nucleases have been found to function most effectively at far higher temperatures, e.g. temperatures in the region of 60 to 75° C.

Thus, insofar as the enzymes disclosed herein possess ring nuclease function and modulate the structure, function and/or activity of cOA (a signalling molecule which is essential to the function of a cellular, type III CRISPR system), they may be used to modulate (destroy, inhibit or ablate) Type III CRISPR systems function and/or the cellular defenses normally potentiated by these systems. As such, the proteins (enzymes) of this disclosure may be described as having an anti-Type III CRISPR activity (or function) and a utility as anti-(type III) CRISPR agents.

Accordingly, the ring nucleases described herein, have significant utility in the field of medicine where they may be used to inactivate, destroy or inhibit medically or clinically important pathogens. For example, any one or more of the ring nucleases described herein may be used in conjunction with phage therapy to destroy or kill clinically important pathogens. One of skill will appreciate that when used in conjunction with phage therapy, the ring nucleases of this disclosure may improve the efficacy of the phage-based therapy or treatment as they can neutralize or inhibit the cellular type III CRISPR system which might otherwise mount an antiviral response preventing the phage (of the phage-based therapy) from replicating.

The ring nucleases disclosed herein may comprise sequences characterised as those belonging to the domain of unknown function 1874 (DUF1874 family). Many DUF protein domains comprise or exhibit a specific and/or unique protein fold. The ring nucleases disclosed herein may comprise a structure, architecture and/or structural fold characteristic of the DUF1874 family. Indeed, the inventors suggest that the DUF1874 sequences with ring nuclease activity, possess a conserved catalytic site for the degradation of cOA molecules, involving a key catalytic histidine residue. More specifically, members of the DUF1874 family with sequences comprising a “GH” active site motif, have ring nuclease activity.

It should be noted that a domain of unknown function (DUF) defines a protein domain (usually based on a highly conserved region) that has no characterised function. Many DUFs are collected together in the Pfam databases and identified by the acronym DUF followed by an identification number. As such, each DUF family represents a well-recognised group, class or family of proteins. DUF 1874 represents one such family (see, for example, http://pfam.xfam.org/family/PF08960 “Family: DUF1874”).

In view of the above, the invention provides the use of a DUF1874 sequence as ring nuclease enzymes. As stated, a useful DUF1874 sequence may comprise one or more of the features selected from the group consisting of:
(i) a conserved catalytic site (for the degradation of cOA molecules);
(ii) a key catalytic histidine residue; and
(iii) a GH active site motif.

Ring nucleases derived from the same or similar source (e.g. ring nucleases derived from viruses (e.g. archaeal viruses)) may comprise more closely related sequences and/or structures than those derived from different sources (e.g. ring nucleases derived from archaeal viruses and ring nucleases derived from bacteria or other microorganisms such as archaea (or proviral or prophage sequences thereof). Despite these differences and as stated above, the present inventors have identified a common motif which may be shared between the newly identified ring nuclease sequences irrespective of the source from which they are derived. The common motif may be referred to herein as a “consensus” motif or sequence and is shown below as SEQ ID NO: 1.

Xaa¹⁻GH-Xaa² SEQ ID NO: 1

As stated, SEQ ID NO: 1 may be a sequence classed as a DUF1874 sequence.

Within SEQ ID NO: 1 each of Xaa¹ and Xaa² independently represent an amino acid sequence flanking a conserved “GH” active site motif. Each of Xaa¹ and Xaa² may (independently) represent any number of amino acids (for any number between about zero and 50).

“Xaa¹” may comprise any number of amino acid residues between 1 and 50, for example 2, 5, 10, 15, 20, 25, 30, 31, 32, 33, 34, 35, 36, 37, 38, 39, 40, 41, 42, 43, 44, 45, 46, 47, 48 or 49 amino acid residues.

“Xaa²” may comprise any number of amino acid residues between zero and 80, for example 2, 5, 10, 15, 20, 25, 30, 35, 40, 45, 50, 55, 60, 65, 66, 67, 68, 69, 70, 71, 72, 73, 74, 75, 76, 77, 78 or 79 amino acid residues.

In addition, the inventors have identified the consensus sequence of SEQ ID NO: 2.

Xaa_{A}-N-Xaa_{B}-GH-Xaa_{C}-NR-Xaa_{D}-R-Xaa_{E}-E-Xaa_{F} SEQ ID NO. 2

Wherein each of Xaa_{A}, Xaa_{B}, Xaa_{C}, Xaa_{D}, Xaa_{E} and Xaa_{F} independently represent the amino acid sequence between each conserved residue. Each of Xaa_{A} to Xaa_{F} may (independently) represent any number of amino acids (for example zero, one, two . . . ten etc.).

As will be appreciated, the residues shown in bold are highly conserved regions of the consensus sequence and represent a common motif that is shared between the ring nuclease sequences disclosed herein.

For example:

“Xaa_{A}” may comprise any number of amino acid residues between 1 and 10 or between 4 and 7.

“Xaa_{B}” may comprise any number of amino acid residues between 25 and 50, or between 30 and 45, or between 33 and 41.

“Xaa_{C}” may comprise any number of amino acid residues between 10 and 25, or between 15 and 20, or may comprise 17 amino acid residues.

“Xaa_{D}” may comprise any number of amino acid residues between 10 and 25, or between 15 and 20, or may comprise 18 or 19 amino acid residues.

“Xaa_{E}” may comprise any number of amino acid residues from zero up to 10, or up to 5, or may comprise 2 amino acid residues.

“Xaa_{F}” may comprise any number of amino acid residues between 5 and 50, or between 10 and 40, or may comprise between 15 and 35 amino acid residues.

As will be appreciated, the residues shown in bold are highly conserved regions of the consensus sequence and represent a common motif that is shared between the ring nucleases disclosed herein. In particular, the region surrounding and/or neighbouring the histidine residue may be highly conserved throughout the ring nuclease family disclosed herein. For example, in some instances, Xaa_{C} may comprise 17 amino acid residues, Xaa_{D} may comprise 18 or 19 amino acid residues and Xaa_{E} may comprise 2 amino acid residues.

In some instances, Xaa_{C} may be or comprise:

Xaa_{C1}-U¹-Xaa_{C2}

wherein

Xaa_{C1} may comprise any number of amino acid residues between 1 and 10, for example, 2 amino acid residues;

U¹ may be T, A, S or G, for example, U¹ may be T or S; and

Xaa_{C2} may comprise any number of amino acids between 1 and 20, for example 14 amino acid residues.

In some instances, Xaa_{D} may be or comprise:

Xaa_{D1}-U²-Xaa_{D2}

wherein

Xaa_{D1} may comprise any number of amino acid residues between 5 and 15, for example, 8 amino acid residues;

U² may be D or Q, for example, U² may be D; and

Xaa_{D2} may comprise any number of amino acids between 5 and 20, for example 9 or 10 amino acid residues.

In some instances, Xaa_{F} may be or comprise:

U³-Xaa_{F1}

wherein

U³ may be G or N, for example, U² may be G; and

Xaa_{F1} may comprise any number of amino acid residues between 5 and 50, or between 10 and 40, or may comprise between 15 and 35 amino acid residues.

Accordingly, in some instances, the consensus sequence or motif of SEQ ID NO. 2 may be denoted as SEQ ID. NO. 3:

Xaa_{A}-N-Xaa_{B}-GH-Xaa_{C1}-U¹-Xaa_{C2}-NR-Xaa_{D1}-U²-Xaa_{D2}-R-Xaa_{E}-E-U³-Xaa_{F1} SEQ ID. NO. 3

wherein each of the Xaa_{A} to Xaa_{F} and U¹ to U³ groups are as defined above and herein.

The ring nucleases disclosed herein may possess a high level of activity and/or potency and/or a broad specificity. Such ring nucleases may act to inhibit, destroy, ablate and/or breakdown cOA activity, structure and/or function more rapidly. In some cases, ring nucleases derived from a virus may degrade cOA molecules more rapidly and/or may have the ability to degrade one or more cOA molecules (e.g. one or more cOA molecules selected from cA₃ to cA₆). By way of further example, the ring nucleases disclosed herein (e.g. those derived from viruses or bacteriophages) may catalyse the breakdown and/or degradation of cA₄ and/or cA₆.

Accordingly, ring nucleases having higher levels of activity and/or potency and/or broad specificity may be further defined by reference to consensus SEQ. ID NO 4 or 5.

Xaa₁-N-Xaa₂-GH-Xaa₃-T-Xaa₄-NR-Xaa₆-D-Xaa₆-R-Xaa₇-EG-Xaa₈ SEQ ID NO: 4

or

Xaa₁-N-Xaa₂-GH-Xaa₃-S-Xaa₄-NR-Xaa₆-D-Xaa₆-R-Xaa₇-EG-Xaa₈ SEQ ID NO: 5

Wherein each of Xaa₁, Xaa₂, Xaa₃, Xaa₄, Xaa₅, Xaa₆, Xaa₇ and Xaa₈ independently represent the amino acid sequence between each conserved residue. Each of Xaa₁ to Xaa₈ may (independently) represent any number of amino acids (for example zero, one, two . . . ten etc).

As will be appreciated, the residues shown in bold are highly conserved regions of the consensus sequence and represent a common motif that is shared between the ring nuclease enzyme sequences disclosed herein.

For example:

“Xaa₁” may comprise any number of amino acid residues between 1 and 10 or between 4 and 7.

“Xaa₂” may comprise any number of amino acid residues between 25 and 50, or between 30 and 45, or between 33 and 41.

“Xaa₃” may comprise any number of amino acid residues from zero up to 10, or from zero up to 5, or may comprise 2 amino acid residues.

“Xaa₄” may comprise any number of amino acid residues from zero up to 20, or between 5 and 20, or may comprise 14 amino acid residues.

“Xaa₅” may comprise any number of amino acid residues from zero up to 20, or between 5 and 10, or may comprise 8 amino acid residues.

“Xaa₆” may comprise any number of amino acid residues from zero up to 20, or between 5 and 15, or may comprise between 9 and 10 amino acid residues.

“Xaa₇” may comprise any number of amino acid residues from zero up to 10, or up to 5, or may comprise 2 amino acid residues.

“Xaa₈” may comprise any number of amino acid residues between 10 and 50, or between 15 and 40, or may comprise between 19 and 31 amino acid residues.

The total number of amino acid residues in any one of the sequences (SEQ ID NO: 1, 2, 3, 4 or 5) may be between 80 and 150, or between 90 and 130 or between 100 and 125 residues.

In some instances, Xaa₁ may be or comprise:

Xaa₁ₐ-X¹X²-Xaa_{1b}

wherein

Xaa₁ₐ may comprise any number of amino acid residues between zero and 10, for example, between 1 and 4;

X¹ may be Y or F;

X² may be L or I; and/or

Xaa_{1b} may comprise any number of amino acids between 0 and 10, for example 1 amino acid residue.

In some instances, Xaa₂ may be or comprise:

X³-Xaa₂ₐ-X⁴-Xaa₂b-X⁵-Xaa_{2b}-X⁶X⁷

wherein:

X³ may be A, S or G;

Xaa₂ₐ may comprise any number of amino acid residues between zero and 10, between 1 and 5, or 4 amino acid residues;

X⁴ may be I, M, L or F;

Xaa_{2b} may comprise any number of amino acid residues between zero and 20, or between 5 and 15, or 11 or 12 amino acid residues;

X⁵ may be I, L or V;

Xaa_{2c} comprises any number of amino acid residues between zero and 40, or between 5 and 25, or between 10 and 20 amino acid residues;

X⁶ may be S or A; and/or

X⁷ may be I or V.

In some instances, Xaa₃ may comprise 2 amino acid residues.

In some instances, Xaa₄ may be or comprise:

Xaa₄ₐ-X⁸-Xaa_{4b}-X⁹-Xaa_{4b}-X¹⁰-Xaa_{4d}

wherein

Xaa₄ₐ may comprise any number of amino acid residues between zero and 5, between 1 and 4, or 3 amino acid residues;

X⁸ may be I, V or L;

Xaa_{4b} may comprise any number of amino acid residues between zero and 5, or between 1 and 4, or may comprise 2 amino acid residues;

X⁹ may be L or I;

Xaa_{4c} may comprise any number of amino acid residues between zero and 10, or between 1 and 5, or may comprise 4 amino acid residues;

X¹⁰ may be L, I, V or F; and/or

Xaa_{4d} may comprise any number of amino acid residues between zero and 5, or between 1 and 4, or may comprise 2 amino acid residues.

In some instances, Xaa₅ may be or comprise:

Xaa₅ₐ-X¹¹-Xaa_{5b}

wherein

Xaa₅ₐ may comprise any number of amino acid residues between zero and 5, or between 1 and 4, or may comprise 2 amino acid residues;

X¹¹ may be I or V; and/or

Xaa_{5b} may comprise any number of amino acid residues between zero and 10, or 1 and 8, or may comprise 5 amino acid residues.

In some instances, Xaa₆ may be or comprise:

Xaa₆ₐ-X¹²-Xaa_{6b}

wherein

Xaa₆ₐ may comprise any number of amino acid residues between zero and 10, or between 1 and 8, or may comprise 6 or 7 amino acid residues;

X¹² may be I, L, V or M; and/or

Xaa_{6b} may comprise any number of amino acid residues between zero and 5, or between 1 and 4, or may comprise 2 amino acid residues.

In some instances, Xaa₇ may be or comprise:

X¹³-Xaa₇ₐ

wherein

X¹³ may be L or I; and/or

Xaa₇ₐ may comprise any number of amino acid residues between zero and 5, or between 1 and 4, or may comprise 1 amino acid residue.

In some instances, Xaa₈ may be or comprise:

Xaa₈ₐ-X¹⁴X¹⁵-Xaa_{8b}

wherein

Xaa₈ₐ may comprise any number of amino acid residues between zero and 5, for example, between 1 and 4, or may comprise 1 amino acid residue;

X¹⁴ may be I or V or may be absent;

X¹⁵ may be V, I, or L or may be absent; and/or

Xaa_{8b} may comprise any number of amino acid residues between 5 and 50, for example, between 10 and 40, or may comprise between 15 and 30 acid residues.

It will be appreciated that the amino acid residues designated as X¹ to X¹⁵ (and also those designated as U¹ to U³) may tolerate some substitution with minimal (or no) loss in activity. For example, the residues at these positions may be substituted with alternative amino acid residues showing similar chemical properties. By way of example, Leucine (L), an amino acid containing a non-polar side chain, may be substituted with any one of the other amino acids generally considered to comprise a non-polar side chain (e.g. Glycine (G), Alanine (A), Valine (V), Isoleucine (I), Methionine (M), Proline (P), Phenylalanine (F) and Tryptophan (W)). Similar substitutions may be made to one or more of the residues X¹ to X¹⁵ and U¹ to U³ within the groups of those amino acids comprising an uncharged polar side chain, an acidic side chain or a basic side chain.

Additionally, or alternatively, analogues of the various peptides described herein may be produced by introducing one or more conservative amino acid substitutions into the primary sequence. One of skill in this field will understand that the term “conservative substitution” is intended to embrace the act of replacing one or more amino acids of a protein or peptide with an alternate amino acid with similar properties and which does not substantially alter the physico-chemical properties and/or structure or function of the native (or wild type) protein. Analogues of this type are also encompassed with the scope of this disclosure.

In particular, the following sequences have been identified as providing ring nuclease enzymes with an ability to modulate (breakdown and/or destroy) cyclic oligoadenylate (cOA) structure, function and/or activity.

THSA-485A (orf Tsac_2833)
(SEQ ID NO. 6)
MFIANAFSLQMLSQFPAHIDIEEVATSAVAKLDLQSAIGHADTAVVLS
GILGKDIESNRVNVQLQPGDSLIVAQLMGGRLPEGSTTLPAGFSFKFF
KVTVQA
SIRV1_gp 29 (orf 114a)
(SEQ ID NO. 7)
MNKVYLANAFSINMLTKFPTKVVIDKIDRLEFCENIDNEDIINSIGHD
STIQLINSLCGTTFQKNRVEIKLEKEDKLYVVQISQRLEEGKILTLEE
ILKLYESGKVQFFEIIVD
STIV_B116
(SEQ ID NO. 8)
MGKVFLTNAFSINMLKEFPTTITIDKLDEEDFCLKLELRLEDGTLINA
IGHDSTINLVNTLCGTQLQKNRVEVKMNEGDEALIIMISQRLEEGKVL
SDKEIKDMYRQGKISFYEVW
AFV3_109
(SEQ ID NO. 9)
MLYILNSAILPLKPGEEYTVKAKEITIQEAKELVTKEQFTSAIGHQAT
AELLSSILGVNVPMNRVQIKVTHGDRILAFMLKQRLPEGVVVKTTEEL
EKIGYELWLFEIQ
ARV1-gp13
(SEQ ID NO. 10)
MLYILNAQITP-FEGAQATFVERRIDVNEAKKIVNSQPFVSAVGHAAT
AQLLSKLLDASIPTNRTQVFLKPGDMALAIVLKSRIPEGVVLDEQAIR
NIGFEIVVIERVS
SIFV_118
(SEQ ID NO. 11)
MLYILNSATLPLKPGKEYVIHAKELTIEEAKELLENERFISAVGHEAT
AKMLTNIFDVEIPMNRIQIFLDDGDKLLSIILKTRLEEGKVIKTVEEL
EQIGYNIWLFEVVTYEHNVKYE
SMV4_113
(SEQ ID NO. 12)
MTVYLANAFSPSMLNKLPSAVEFQRVDQKEFCEAIHHGVSNAIGHKGT
IEFVNTLCNTNLQTNRVEIKAGINDVIYIIVLGFRLEEGKVLSAGEVQ
KAYDEGKVLLLKAIIGK
SIFV2_gp23
(SEQ ID NO. 13)
MLYILNSATLPLKPGKEYVIHAKELTIEEAKELLENERFISAVGHEAT
AKMLTNIFGVEISMNRIQIFLDDGDKLLSIILKTRLEEGKVIKTVEEL
EQIGYNIWLFEVVTYVHNAKYE
AFV1_116
(SEQ ID NO. 14)
MGSYLLNGFSPAMLASGHSVVFFNQIPDVMLCGALTSDELVNAIGHKS
TINLINRICQTNLKENRIQVMLQDGDEAFIVVVTERLEEGKVLSDEEI
TKMFEDGKIKIYYARVHSVV
Yddf
(SEQ ID NO. 15)
MEIAFLNSLVVTSPGFYKAEKITLDEVKQWLKHYDGRYKSFIGHKSTA
QFLQKLLGIRIEQNRKTFRHMKYQKAICFSLYERYPENVLLTQRDLEK
ARYQFYLLTRLD
Sequence WP_087145848.1 from the bacterium
Crenothrix polyspora:
(SEQ ID NO. 16)
MTLFIINAPILTSYGDWRFEGPLSIDKTRKLLREGFTSAIGHAASAEM
LARLLAMDIPVNRIAITMEAGDRALILRLLQRLPEGKVLNHHEMMATP
FELALLTKLK
Sequence from Nitrosomonas_marina:
(SEQ ID NO. 17)
MLYLINSPILTSYGDWQFSGPLTVAEAKSRLSNDFISAIGHQSGAAFL
SALLDIEIPVNRIEINMKPGDSALVLRLKSRLPEGKVLTHDEMQQIPY
ELGWLVRVQ
Sequence from Methylomagnum ischizawi:
(SEQ ID NO. 18)
MALIYVINSPVLTGYGLWRFEGPLAESAARELLAGGFVSALGHAGAAR
FLSARLGLGIPVNRVRVELQPGDRALVLRLLERLPENRALSAAEMEEL
PFELGLLTRLE
Sequence from Desulfurella_multipotens:
(SEQ ID NO. 19)
MVYLLNGPILTDFGLYRYKKISILQAKKILKENKFVSAIGHEATAIFL
SELLELDIKYNRIAIKMKQGDLAIVFHLLTRLKEGQVLNIKELCSKDY
TLGILKRLE
ATV_gp06
(SEQ ID NO. 20)
MGVWSVVLYLLNTLIVPFRDERAKFEIERVSAEEAKKIIQMHNSQFVS
AIGHSASANALSLLLGVAVPVNRTEVFFNVGDEAIAMALKKRLAEGQV
LRTVQELEAVGFDLYYIKRVQ
Synechococcus phage S-CBWM1
(SEQ ID NO. 21)
MACCVVPKGAPGLWSVVEISLEEVIQDLEEGEFISTIGHPSSAHILET
LTGFPFEACRREADPRPGDEFYCFILNSRAPEGKILDEHEIYKIGFSF
RKMTYVLGKIPTAPD
Fusobacterium phage Fnu1
(SEQ ID NO. 22)
MTIGILNTPILTGEGTYKLSNITLEQAQKLVNENEFISYIGHQATAEI
ISILLGTEVPMNRGQFKQEVGQKAIIFKLKSRLLEGQILLTIQEIEEI
GYEFQLLERKN
Hydrogenobaculum phage 1
(SEQ ID NO. 23)
MLYVLNSLIVPVDFQNKQGYIVSLWKIDLETARKIVREMPFTSAVGHE
ATAKVLSELLGVEISFNRITVKMKEGDAGLHFVLRTRLPEGKVLSEEE
LRQLDFDLVLSRVS
ICEBs1 Yddf
(SEQ ID NO. 24)
MEIAFLNSLVVTSPGFYKAEKITLDELKHYDGRYKSFIGHKSTAQFLQ
KLLGIRIEQNRKTFRHMKYQKAICFSLYERYPENVLLTQRDLEKARYQ
FYLLTRLD

The disclosure further embraces functional variants, derivatives, portions or fragments of any of the sequences disclosed herein as SEQ ID NOS: 1-24.

The disclosure further provides nucleic acid sequences encoding any one of SEQ ID NOS: 1-24 described herein and functional fragments thereof.

Using the sequence information described herein, PCR, cloning and recombinant techniques can be used to synthesise copies of any of the nucleic acid or peptide/protein sequences described herein, including those provided by SEQ ID NOS: 1-24. For example, oligonucleotide primers which bind to regions (for example short 10-20 base pair and/or GC rich regions) of those nucleic acid sequences encoding SEQ ID NOS: 1-24 may be used to amplify specific ring nuclease nucleic acid sequences, which amplified sequences are then cloned and expressed in order to generate ring nuclease enzyme for purification. Thus, the disclosure provides recombinant nucleic acid sequences encoding any of the ring nuclease sequences described herein—including any of those sequences provided by SEQ ID NOS: 1-24 and functional fragments thereof.

One of skill will understand that the term “functional” relates to the ring nuclease activity of any of the full or complete enzymes described herein. Thus a functional variant, derivative, fragment or portion, is any variant, derivative, fragment or portion of any one of the sequences described herein that exhibits ring nuclease activity and/or an ability to modulate the function, structure and/or activity (i.e. promote, catalyse or stimulate the breakdown and/or destruction of) cOA.

As will be appreciated, the ring nucleases described herein may find use in medicine, as a medicament, medical treatment and/or in therapy. Thus, one aspect provides a ring nuclease enzyme comprising a sequence provided by SEQ ID NOS: 1-24 or a functional fragment thereof, for use:
(i) in medicine;
(ii) in therapy; and/or
(iii) as a medicament

A ring nuclease of this disclosure (i.e. a ring nuclease comprising a sequence provided by any one of SEQ ID NOS: 1-24 or a functional fragment thereof) may be used in the treatment and/or prevention of pathogenic infections. A ring nuclease of this disclosure may be used as an antimicrobial agent. Any of the disclosed ring nucleases may be used in the treatment and/or prevention of bacterial infections, including the treatment and/or prevention of infections arising from antibiotic-resistant pathogenic bacteria.

As stated, the disclosed ring nucleases have been shown to modulate the structure, function and/or activity of cOA, a signalling molecule associated with the initiation of an antiviral response in Type III CRISPR systems. Thus, the ring nucleases of this disclosure may be used in the treatment or prevention of any condition arising from (or caused and/or contributed to by) a pathogen encoding or harbouring a Type III CRISPR system.

Representative examples of pathogenic bacteria known to encode a type III CRISPR system are indicated in Table 1.

TABLE 1
Pathogenic bacteria with a type III CRISPR system

| Species | Disease |
|---|---|
| Mycobacterium tuberculosis | Tuberculosis |
| Neisseria meningitidis | bacterial meningitis and sepsis |
| Neisseria mucosa | endocarditis |
| Staphylococcus aureus | sepsis and toxic shock |
| Streptococcus pyogenes | sepsis, pharyngitis |
| Streptococcus mutans | tooth decay |
| Pectobacterium carotovorum | Ubiquitous plant pathogen |
| Dickeya dadantii | Crop pathogen |

Accordingly, it will be appreciated that the ring nucleases described herein (e.g. comprising a sequence provided by any one of SEQ ID NOS: 1-24 or a functional fragment thereof) may find use in the treatment and/or prevention of diseases and/or conditions caused or contributed to by one or more of the bacterial species listed in table 1. Additionally, or alternatively, the ring nucleases described herein may find use in the treatment and/or prevention one or more of the diseases listed in Table 1. The pathogenic bacteria (and associated diseases) listed in Table 1 are representative (and not limiting) examples of pathogens encoding a type III CRISPR system. However, as stated above, any pathogen encoding or harbouring a Type III CRISPR system may be targeted by the ring nucleases of this disclosure. Consequently, any disease or condition arising from (or caused and/or contributed to by) a pathogen encoding or harbouring a Type III CRISPR system may be treated and/or prevented by the ring nucleases of this disclosure.

Thus, in further aspects, the disclosure provides a ring nuclease enzyme comprising a sequence provided by SEQ ID NOS: 1-24 or a functional fragment thereof, for use:
(i) in treating and/or preventing diseases and/or conditions caused or contributed to by one or more of the bacterial species listed in table 1; and/or
(ii) in treating and/or preventing one or more of the diseases listed in Table 1.

In some instances, any one or more of the disclosed ring nuclease(s) may be used in phage therapy. For example, any of the disclosed ring nucleases may be administered in combination with a phage. It should be noted that where a ring nuclease of this disclosure (for example a ring nuclease comprising a sequence provided by SEQ ID NOS: 1-24 or a functional fragment thereof) is to be administered in combination with a phage, the ring nuclease may be administered before the phage and/or after the phage and/or concurrently (together with) the phage.

One of skill will appreciate that while the ring nucleases of this disclosure have many uses, so too may ring nuclease modulators. As used herein, “ring nuclease modulators” may refer to agents or molecules that modulate the function, structure and/or activity of any of the ring nucleases described herein. For example, suitable modulators may have ring nuclease agonistic, antagonistic or inhibitor function.

An agent which acts to inhibit the function, structure and/or activity of a ring nuclease may be referred to as a ring nuclease inhibitor. Within the context of this invention, useful ring nuclease inhibitor molecules may be referred to as ring nuclease inhibitors (RNi). One of skill will appreciate that any given RNi may be used to render a cell resistant or immune to viral/phage infection. For example, a RNi could be used to prevent any ring nuclease of this disclosure (for example a phage or viral encoded ring nuclease) from incapacitating a type III CRISPR system.

Ring nuclease modulators, including the abovementioned RNi(s) may be used in microbiological processes to prevent industrially important bacteria from succumbing to phage attack. In other words, a ring nuclease modulator (for example a RNi) may be used in an industrial process comprising the use of microorganisms, wherein the ring nuclease modulator serves to protect the microorganism from viral infection and/or attack.

Accordingly, the disclosure provides a method of using a ring nuclease modulator, for example a RNi, in an industrial microbial process and/or system.

An industrial process and/or system may involve, comprise or rely on the use of a microorganism possessing or comprising a Type III CRISPR system. In such cases phage expressing any of the ring nucleases described herein may infect the microorganism and (via ring nuclease activity) neutralise the Type III CRISPR system thereby ablating any associated antiviral effects. This would have an adverse impact on any industrial process as the microorganism component of the process would become inactivated/destroyed by the phage. Use of a ring nuclease modulator, for example a RNi, would prevent, limit or inhibit ring nuclease activity and permit any Type III CRISPR system expressed by the microorganism to induce an antiviral response, defending the microorganism against phage attack. Without wishing to be bound by theory, a ring nuclease inhibitor may prevent and/or inhibit the growth, replication and/or spread of a phage in the industrial process and/or system, and/or may be used to kill the phage in the industrial process and/or system.

A ring nuclease inhibitor may be contacted with a microorganism possessing or relying on a Type III CRISPR system. The ring nuclease inhibitor may be introduced into the microorganism and/or added during one or more steps of the (industrial) process.

Representative examples of industrially important microorganisms possessing a Type III CRISPR system include, but are not limited to, Streptococcus thermophilus (useful in the manufacture of yoghurt) and Clostridium beijerinckii (useful in the production of butanol, acetone, isopropanol, valuable chemicals and/or for hydrogen production). Any of the ring nucleases described herein might act to inactivate the type III CRISPR system of these microorganisms. A RNi of this disclosure could be used to inhibit phage ring nuclease activity and preserve Type III CRISPR system function.

Ring nuclease modulators may be identified by any suitable method. For example a ring nuclease modulator screening assay (a “RN modulator assay”) may comprise contacting a potential modulator (e.g. a test agent) with a ring nuclease enzyme (for example a ring nuclease described herein) and monitoring the enzyme for any change (modulation) in function and/or activity. Detecting changes in ring nuclease enzyme function and/or activity indicates that the test agent may be a ring nuclease modulator.

The step of monitoring for and/or detecting any change in the function and/or activity of the ring nuclease may comprise comparing an output from the screening assay carried out in the presence of a potential modulator to a standard, reference or baseline level of function and/or activity of the ring nuclease. Any difference between the output and the standard, reference or baseline level of ring nuclease activity and/or function may indicate that the test agent is a ring nuclease modulator.

The standard, reference or baseline level of function and/or activity of the ring nuclease may be obtained by monitoring for and/or detecting an output from a ring nuclease in the absence of any potential modulator. By way of example, the method may comprise monitoring and/or detecting any change in the levels of cOA and/or any change in cOA activity, structure and/or function to provide a standard, reference or baseline function and/or activity for the ring nuclease.

A useful ring nuclease modulator assay may comprise any one of the ring nucleases described herein. Further the output of such an assay (i.e. ring nuclease function or activity in the presence of a test agent) may be compared with a standard, control or normal level of ring nuclease function or activity. As stated, any difference in ring nuclease function or activity (in the presence of a test agent) might suggest that the test agent is a ring nuclease modulator.

The present disclosure also extends to the use of the ring nucleases in molecular biology applications, diagnostic methods and/or diagnostic technology. These uses may be based on the fact that cOA activates a number of enzymes. By way of example, cOA is known to activate certain degradative enzymes. In particular, cOA may be used to activate ribonuclease and/or DNA nuclease which may subsequently lead to the degradation of RNA and/or DNA. One of skill will appreciate that a ring nuclease may be used with such cOA activated enzymes in a process to provide and/or facilitate a controlled degradation of genetic material (e.g. RNA and/or DNA).

A method of controllably degrading a sample of genetic material may comprise contacting the sample with one or more of the degradative enzymes in the presence of cOA and a ring nuclease. The cOA and the ring nuclease may be added sequentially to the sample and the one or more degradative enzymes. Alternatively, the method may comprise one or more cycles of alternating cOA and ring nuclease additions. The use of cOA with a ring nuclease in such a method may provide a tightly regulated method of switching on and off degradative enzymes and/or may provide a controlled degradation of the sample.

As stated, cOA is an important signalling molecule. Therefore, the ability of the ring nucleases to modulate the function, structure and/or activity of cOA may act to disrupt cOA signalling within an organism. By way of example, it is believed that detection of certain RNA species could switch on cOA synthesis leading to changes in gene expression and/or cell metabolism within a cell, optionally via activation of proteins such as CARF domain proteins (CRISPR-associated Rossman Fold domain proteins). Accordingly, the ring nucleases disclosed herein may also find application in the control and/or modulation of gene expression and/or cell metabolism (e.g. by disrupting the cOA signalling).

The disclosure further provides antibodies with specificity or affinity for any of the ring nucleases described herein. Such antibodies may be monoclonal and/or polyclonal. The term “antibodies” further includes antigen binding fragments. Thus, the antibodies of this disclosure may bind (or have specificity or affinity for) one or more ring nuclease epitopes. It will be appreciated that antibodies with affinity for the ring nucleases of this disclosure may be obtained by methods which involve immunising animals (for example rodents) with purified or isolated forms of the ring nucleases described herein. Such methods may, for example, use the recombinant ring nucleases described herein. Further, techniques used to generate monoclonal antibodies (mAbs) are well known and can easily be exploited to generate mAbs specific for any one of the sequences described herein or fragments thereof. Similarly, the processes used to generate polyclonal antibodies are also well established and may be used to generate antibodies specific for epitopes carried by any of the ring nucleases described herein.

The present disclosure also extends to methods for identifying or detecting amino acid and or nucleic acid sequences which potentially encode or provide ring nucleases. Such sequences shall be referred to hereinafter as “ring nuclease sequences”.

A method of identifying or detecting a ring nuclease amino sequence may comprise probing or screening an amino acid sequence for the presence of one or more of the sequences (including consensus sequences and functional “fragments” thereof) described herein, wherein an amino acid sequence found to comprise such a sequence may be a ring nuclease amino acid sequence (i.e. an amino acid sequence which potentially encodes or provides a ring nuclease).

A method of identifying or detecting ring nuclease amino acid sequences may comprise a first step of providing or obtaining an amino acid sequence (which amino acid sequence may be one suspected of harbouring a sequence which provides (or encodes) a ring nuclease). The method may further comprise subjecting the provided or obtained amino acid sequence to a sequencing protocol or procedure so as to determine the primary sequence thereof. A determined primary sequence may then be investigated for the presence of a sequence having a degree of similarity or identity to any of the ring nuclease amino acid sequences described herein. Ring nuclease amino acid sequences may comprise sequences which are anywhere between about 30% and about 100% (for example 40%, 45%, 50%, 55%, 60%, 65%, 70%, 75%, 89%, 85%, 90%, 95%, 96%, 97%, 98% or 99%) homologous or identical to a sequence described herein or a functional fragment, variant or derivative thereof. Homologous or identical ring nuclease amino acid sequences may be identified by comparing any sequence with a library of sequences known to encode ring nucleases.

One of skill will appreciate that antibodies specific for ring nuclease epitopes and in particular epitopes present within any of the ring nucleases disclosed herein, may be used to identify ring nuclease amino acid sequences. Specifically, an antibody with affinity for a ring nuclease epitope may be contacted with any given peptide or protein sequence under conditions which permit binding between the antibody and its epitope. Binding of such an antibody to a peptide or protein sequence indicates that the amino acid sequence may comprise a ring nuclease amino acid sequence. Assays of this type may exploit conjugated antibodies—i.e. antibodies that are conjugated to detectable tags (chemiluminescent, radio labels and the like). Antibodies specific for epitopes present within the ring nucleases described herein are discussed below.

This disclosure may further provide methods of identifying or detecting ring nuclease nucleic acid sequences. In such cases nucleic acid sequences may be probed and/or screened for the presence of ring nuclease nucleic acid sequences which encode any of the sequences described herein (including the consensus sequences and ring nuclease sequences of SEQ ID NOS: 1 to 24 and functional fragments of any of these). Wherein a nucleic acid sequence found to comprise such a sequence may be a ring nuclease nucleic acid sequence (i.e. a nucleic acid sequence which potentially encodes a ring nuclease).

A method of identifying or detecting a ring nuclease nucleic acid sequence may comprise providing or obtaining a nucleic acid sequence (which nucleic acid sequence may be one suspected of harbouring a sequence which encodes a ring nuclease). The method may further comprise subjecting the provided or obtained nucleic acid sequence to a sequencing protocol or procedure so as to determine the sequence thereof. A determined sequence may then be investigated for the presence of a sequence having a degree of similarity or identity to any of the ring nuclease nucleic acid sequences described herein. Those sequences which harbour ring nuclease encoding nucleic sequences may comprise sequences which are anywhere between about 30% and about 100% (for example 40%, 50%, 55%, 60%, 65%, 70%, 75%, 89%, 85%, 90%, 95%, 96%, 97%, 98% or 99%) similar or identical to the various nucleic acid sequences described herein or a functional fragment, variant or derivative thereof.

A method of identifying or detecting a ring nuclease nucleic acid may exploit oligonucleotide probes which bind (under suitable (for example stringent) conditions) to ring nuclease nucleic acid sequences. Suitable probes may be referred to as ring nuclease probes. Suitable probes may comprise oligonucleotides. Useful oligonucleotide probes may comprise sequences which are complementary to sequences (for example short continuous sequences) present in any of the ring nuclease sequences described herein. For example, a useful probe may comprise a sequence complementary to a sequence of about 5 to about 100 (for example about 10, 15, 25, 25, 30, 35, 40, 45, 50, 55, 60, 65, 70, 75, 80, 85, 90, 95 or about 99) continuous or contiguous nucleotides of any of the ring nuclease nucleic acid sequences described herein. Thus, a method for identifying or detecting a ring nuclease nucleic acid sequence may comprise a step in which a ring nuclease probe is contacted with a nucleic acid sequence under conditions which permit binding between the probe and any complementary sequence present in the nucleic acid sequence being screened. Binding between the probe and the nucleic acid sequence indicates that the nucleic acid sequence may encode a ring nuclease sequence.

A nucleic acid may also be sequenced and the obtained sequence compared to any of the sequences described, whereupon identification of a degree of identity or similarity between the sequenced nucleic acid sequence and any of the sequences described herein (including any of the consensus sequences), indicates that the nucleic acid sequence might be a ring nuclease nucleic acid sequence.

The degree of (or percentage) “similarity” between two or more (amino acid (or nucleic acid)) sequences may be determined by aligning the sequences and determining the number of aligned residues which are identical or, in the case of a nucleic acid sequence, which are not identical but which differ by redundant substitutions (a “redundant substitution” being a conservative amino acid substitution or one which has no effect upon the function, structure and/or property of the peptide or protein provided by the amino acid sequence or, in the case of a nucleic acid sequence, the amino acid encoded by the codon). A degree (or percentage) “identity” between two or more (amino acid or nucleic acid) sequences may also be determined by aligning the sequences and ascertaining the number of exact residue matches between the aligned sequences and dividing this number by the number of total residues compared—multiplying the resultant figure by 100 would yield the percentage identity between the sequences.

One of skill will appreciate that any of the methods described herein (namely the methods for identifying ring nuclease nucleic and/or amino acid sequences) may be conducted in silico. For example, at least the initial step of screening or probing the amino acid or nucleic acid sequences for a motif may be carried out in silico.

A sequence subjected to any of the (screening) methods described herein may be any suitable sequence. Sequences that may be subject to the methods described herein may be prokaryotic or eukaryotic in origin; they may be derived from microorganisms (for example bacterial and/or viral sequences), fungi, plants and/or animals. Suitable sequences may include deposited sequences (i.e. a sequence deposited within some form of database, for example a publically accessible sequence data base), uncurated deposited sequences, hypothetical protein sequences, unannotated sequences, genomic sequences and the like.

The screening methods, particular those used to identify ring nuclease amino acids, may comprise a further step in which an amino acid sequence identified as potentially encoding or providing a ring nuclease (e.g. a potential candidate ring nuclease) may be subjected to an assay to determine a level or presence of ring nuclease activity. An assay of this type may be referred to as a ring nuclease assay. Any nucleic acid identified as a potential ring nuclease nucleic acid sequence may be cloned and expressed to yield a peptide or protein that can also be subjected to a ring nuclease assay.

Useful ring nuclease assays may comprise the steps of contacting a potential candidate (i.e. a “test agent”) with cOA and monitoring for any modulation (degradation) of cOA structure, function and/or activity and/or any products resulting from the modulation (for example degradation) of cOA structure, function and/or activity. The assay may comprise the steps of contacting a potential test agent with cA4 and/or cA6.

This disclosure also embraces novel ring nucleases identifiable and/or obtainable by any of the methods outlined herein.

The disclosure further provides ring nuclease modulators (for example RNi(s)) identifiable and/or obtainable by any of the methods outlined herein.

### DETAILED DESCRIPTION

The present invention will now be described in detail with reference to the following Figures which show:

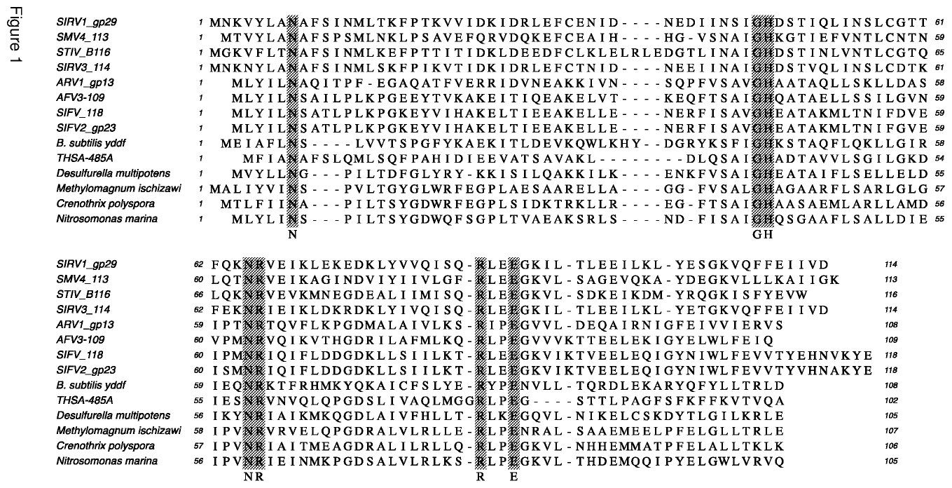

FIG. 1. Multiple sequence alignment of the ring nuclease family, with conserved residues highlighted. Representative archaeal virus proteins are shown, along with the phage protein THSA-485A Tsac_2833 and proteins found in bacterial genomes (Bacillus subtilis, Desuflurella multipotens, Methylomagnum ischizawi, Crenothrix polyspora and Nitrosomonas marina).

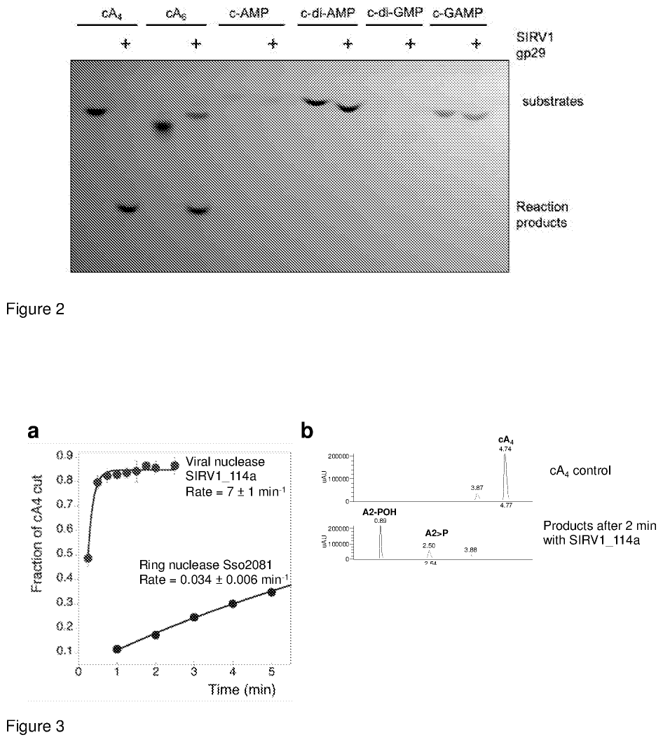

FIG. 2. Degradation of cA₄ and cA₆ in the presence of SIRV1 gp29 (orf 114a).

Thin layer chromatography (TLC) analysis shows that, under multiple-turnover conditions (49 μM protein; 450 μM substrate) at 70° C., SIRV1 gp29 rapidly degrades cA₄ and cA₆, but does not degrade cyclic-AMP, or the cyclic dinucleotides, cyclic-di-AMP, cyclic-di-GMP or cyclic-CAMP.

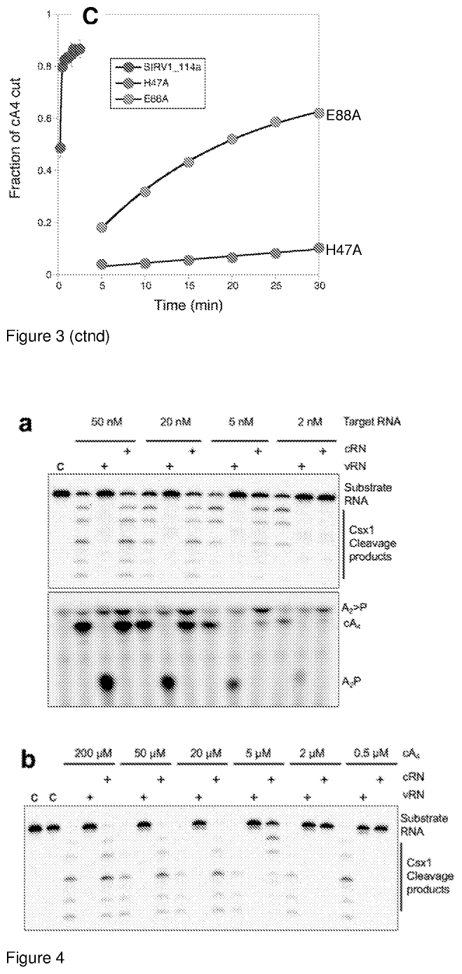

FIG. 3. Analysis of cA₄ degradation by SIRV gp29 (orf 114a).
a) Single-turnover kinetic comparison of cA₄ degradation at 50° C. by ring nuclease SIRV1 gp29 (4 μM dimer) and cellular Ring nuclease Sso2081 (4 μM dimer). The viral protein is 200× more active than the characterised cellular ring nuclease.
b) LC-HRMS analysis demonstrates that SIRV1 gp29 converts cA₄ into linear A2-POH (diadenylate ApAp), with minor amounts of ApA>P (with a 2′, 3′-cyclic phosphate) as an intermediate.
c) Wild type SIRV1 gp29 (orf 114a) cleaves cA₄ with a single turnover rate constant of 7.4 min⁻¹. The H47A and E88A variants have rate constants of 0.00014 and 0.058 min⁻¹, respectively, indicating their important role in catalysis.

FIG. 4. Comparison of the kinetics of the ring nucleases disclosed herein and cellular ring nucleases in deactivating the type III CRISPR defence.
a) Top panel is a phosphor image of denaturing polyacrylamide gel electrophoresis showing activation of HEPN family ribonuclease Csx1 (0.5 μM dimer) and consequent radioactively labelled RNA A1 cleavage in a coupled assay containing type III Csm CRISPR complex carrying A26 CRISPR RNA when challenged by indicated amounts of A26 RNA target to initiate cA₄ synthesis. RNA A1 is not a target of Csm. Each set of three lanes after the control (c) reaction with Csx1 and labelled non-target RNA alone, is first in the absence and then the presence the ring nuclease SIRV1 gp29 (labelled “vRN”) or S. solfataricus cellular ring nuclease (cRN) Sso2081 (2 μM dimer), respectively. Whereas the SIRV1 gp29 ring nuclease is able to degrade all cA₄ generated with up to 50 nM RNA A26 target, the cellular ring nuclease deactivates Csx1 and protects substrate RNA from degradation only when less than 5 nM A26 RNA target is used to initiate cA₄ synthesis. Bottom panel is a phosphor image of thin-layer chromatography with reactions as above but visualizes cA₄ production, by a-ATP incorporation, in the presence of indicated amounts of A26 RNA target and absence or presence of either SIRV1 gp29 or cellular ring nuclease. Csx1 deactivation correlated with complete cA₄ degradation.
b) Denaturing gel electrophoresis visualising activation of Csx1 (0.5 μM dimer) by indicated amounts of HPLC-purified cA₄ (BIOLOG Life Sciences) and its subsequent deactivation when either SIRV1 gp29 ring nuclease (“vRN”) or cellular ring nuclease (2 μM dimer) is present to degrade cA₄. The SIRV1 gp29 ring nuclease degrades 100-fold more cA₄ than the cellular ring nuclease. Control reactions (c) show RNA incubated with SIRV1 gp29 or Csx1 in the absence of cA₄, respectively.

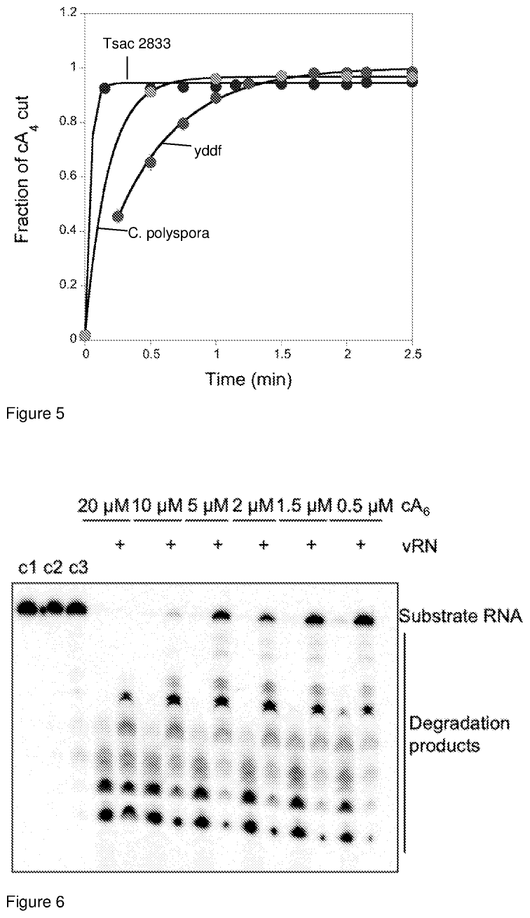

FIG. 5. Comparison of cA₄ cleavage kinetics for three further DUF1874 proteins.

The recombinant ring nuclease proteins encoded by B. subtilis yddf (green), THSA-485A Tsac_2833 (blue) and Crenothrix polyspora (orange) all cleave cA₄ rapidly in vitro.

FIG. 6. Degradation of cA₆ in the presence of THSA-485A Tsac_2833 (deactivating the HEPN nuclease StCsm6′).

Ring nuclease (Tsac_2833, 2 μM dimer) was incubated with the indicated concentration of cA₆ for 60 min at 37° C. The cA₆-activated HEPN family ribonuclease StCsm6′ was then added along with radioactively labelled substrate RNA and incubated for 60 min at 37° C. before denaturing gel electrophoresis and phosphor imaging. cA₆ degradation resulted in protection of the substrate RNA due to deactivation of StCsm6′. Control c1 is RNA in the absence of protein, c2 is RNA incubated with Tsac_2833 and c3 is RNA incubated with StCsm6′ in the absence of cA6 activator. vRN—ring nuclease.

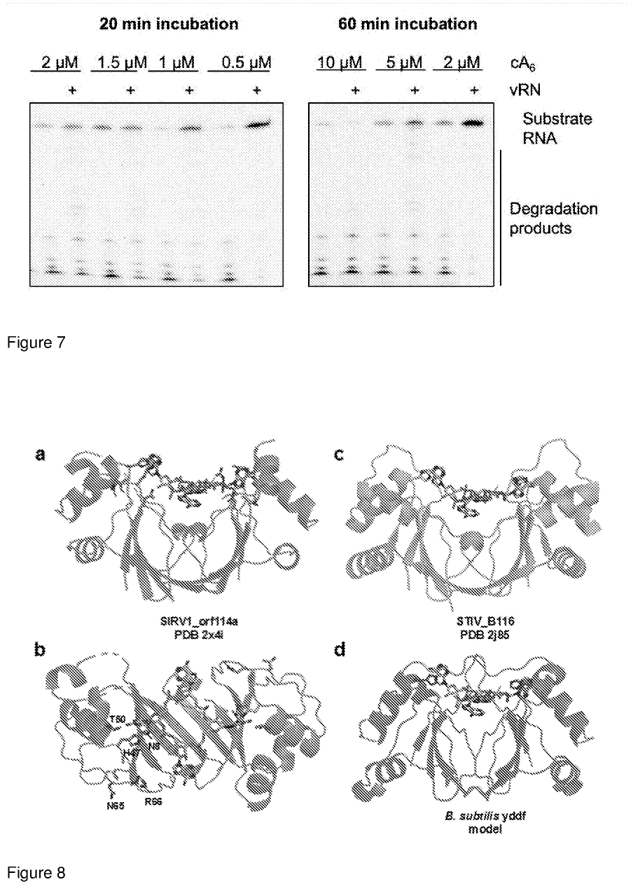

FIG. 7. Degradation of cA₆ in the presence of SIRV1 gp29 (deactivating the HEPN nuclease StCsm6′).

Ring nuclease (SIRV1_114a, 2 μM; labelled “vRN”) was incubated with the indicated concentration of cA₆ for 20 or 60 min at 70° C. and cooled on ice for 5 min. The cA₆-activated HEPN family ribonuclease StCsm6′ was then added along with radioactively labelled substrate RNA and incubated for 60 min at 37° C. before gel electrophoresis. cA₆ degradation resulted in protection of the substrate RNA due to deactivation of StCsm6′.

FIG. 8. Structural comparison of ring nucleases, with cA₄ modelled into the binding site.
a) Structure of SIRV1_114a (PDB 2×41) with modelled cA₄. The subunits are shown in cartoon form and coloured blue and green. Conserved residues are shown.
b) Orthogonal view of SIRV1_114a, looking down on the cA₄ binding site. Conserved residues are labelled.
c) Structure of the STIV_B116 dimer (PDB 2j85), with cA₄ modelled and the conserved histidine side chain indicated.
d) Model of the B. subtilis YddF protein, which has a prophage origin (model generated using Phyre (Kelley et al, Nature protocols, 2009, 4(3): 363-371).

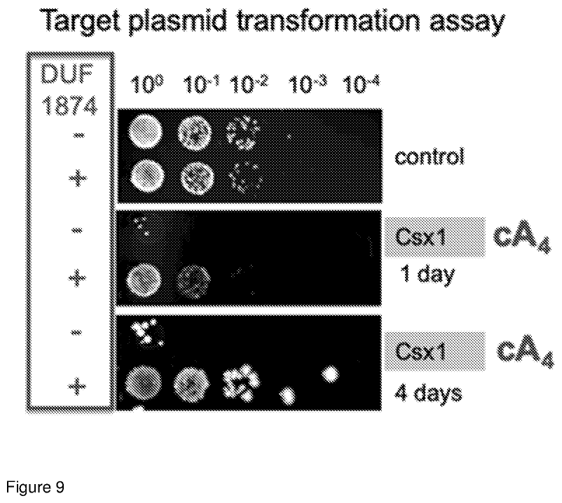

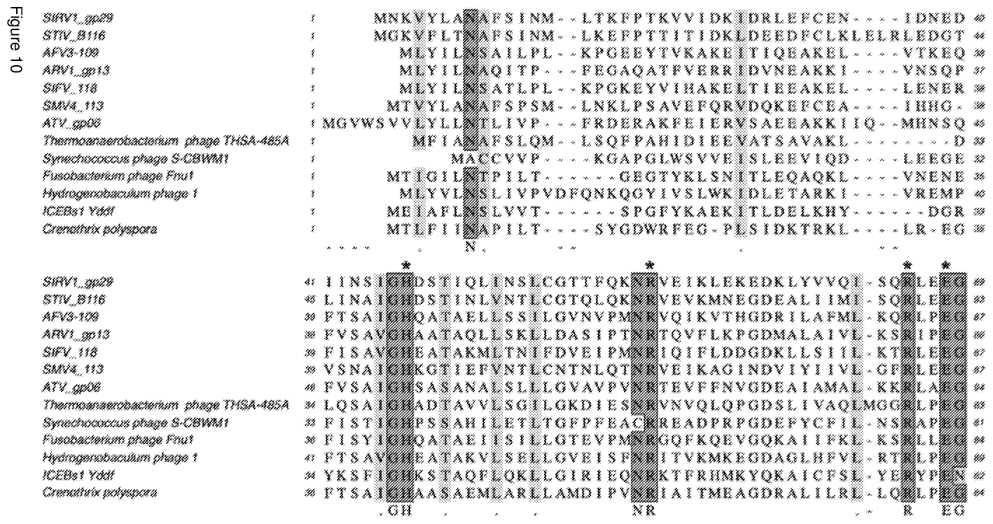

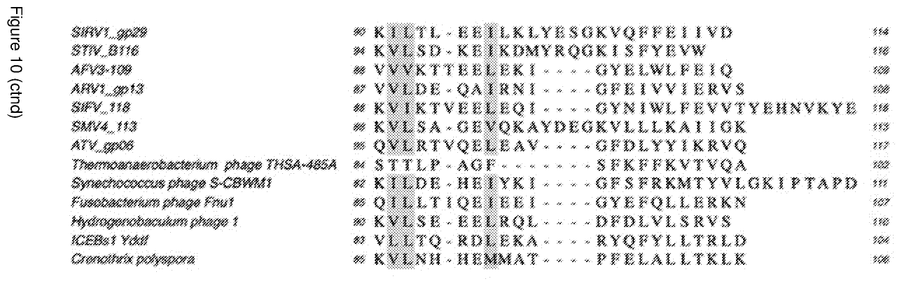

FIG. 9. Ring nuclease THSA-485A Tsac_2833 overcomes type III CRISPR immunity in vivo.

Plasmid transformation assay (1 and 4 day's growth) using a plasmid with a match to a spacer in the CRISPR array. If the plasmid was successfully targeted by the CRISPR system, fewer transformants were expected. Cells using a cA₄-based (Csx1) system only reduced plasmid transformation when the DUF1874 protein Tsac_2833 was not present, suggesting that the DUF1874 ring nuclease was effective in neutralising a cA₄-mediated CRISPR defence. The control strain lacked cOA-dependent ribonucleases. These results are representative from two biological replicates with four technical replicates each (n=8).

FIG. 10. Multiple sequence alignment of the ring nuclease family, with conserved residues highlighted.

This multiple sequence alignment includes representative archaeal virus proteins (SIRV1, STIV, AFV3, ARV1, SIFV, SMV4 and ATV), along with the phage proteins THSA-485A Tsac_2833, Synechococcus phage S-CBWM1, Fusobacterium phage Fnu1 and Hydrogenobaculum phage 1, and proteins found in bacterial genomes (ICEBs1 protein from Bacillus subtilis, and the Crn2 protein from Crenothrix polyspora). Light and dark grey shading indicate regions of partial and strong sequence conservation respectively.

### METHODS

The methods used herein are described below.

Cloning and Purification

For cloning, synthetic genes (g-blocks) were purchased from Integrated DNA Technologies (IDT), Coralville, USA, and cloned into the vector pEhisV5spacerTev between NcoI and BamHI sites. Competent DH5a (Escherichia coli) cells were then transformed with the construct and sequence integrity confirmed by sequencing (Eurofins Genomics). Plasmid was then transformed into Escherichia coli C43 (DE3) cells for protein expression. For expression of SIRV1 gp29, YddF, THSA-485A Tsac_2833, and Crenothrix polyspora CRENPOLY SF2_1390015, 2 L of culture was grown at 37° C. to an OD₆₀₀ of 0.8 with shaking at 180 rpm. Protein expression was then induced with 0.4 mM isopropyl β-D-1-thiogalactopyranoside (IPTG) and cells grown at 25° C. overnight before harvesting by centrifugation at 4000 rpm (Beckman Coulter Avanti JXN-26; JLA8.1 rotor) at 4° C. for 15 min.

For protein purification the cell pellet was resuspended in four volumes equivalent of lysis buffer containing 50 mM 2-amino-2-(hydroxymethyl)-1,3-propanediol (Tris)-HCl 7.0, 0.5 M NaCl, 10 mM imidazole and 10% glycerol supplemented with mini EDTA-free protease inhibitor tablets (Roche; 1 tablet per 20 ml buffer) and lysozyme (1 mg/ml). Cells were then lysed by sonicating six times 1.5 min with 1.5 min rest intervals on ice at 4° C., and the lysate was ultracentrifuged at 40,000 rpm (70 Ti rotor) at 4° C. for 45 min. The lysate was then filtered using a 0.45 μm syringe filter and loaded onto a 5 ml HisTrap FF column (GE Healthcare) equilibrated with wash buffer containing 50 mM Tris-HCl pH 7.0, 0.5 M NaCl, 30 mM imidazole and 10% glycerol. Unbound protein was washed away with 20 column volumes (CV) of wash buffer prior to elution of his-tagged protein using a linear gradient of elution buffer containing 50 mM Tris-HCl pH 7.0, 0.5 M NaCl, 0.5 M imidazole and 10% glycerol. SDS-PAGE was then carried out to identify fractions containing the protein of interest, and the relevant fractions were pooled and concentrated using an ultracentrifugal concentrator (MERK). The his-tag was removed by incubating concentrated protein overnight with Tobacco Etch Virus (TEV) protease (1 mg per 10 mg protein) while dialysing in buffer containing 20 mM Tris-HCl pH 7.0, 0.5 M NaCl and 1 mM DTT. The his-tag removed protein was then isolated using a 5 ml HisTrapFF column, eluting the protein using 2 CV wash buffer. His-tag removed protein was further purified by size-exclusion chromatography (S200 16/60; GE Healthcare) in buffer containing 20 mM Tris-HCl pH 7.0, 0.5 M NaCl and 1 mM DTT using an isocratic gradient. After SDS-PAGE, fractions containing protein of interest were concentrated and protein was aliquoted and stored at −80° C.

Radiolabelled cA₄ Cleavage Assays

Cyclic oligoadenylate (cOA) was generated by incubating 120 μg Sulfolobus solfataricus (Sso) Type III-S (Csm) complex with 5 nM α-³²P-ATP, 1 mM ATP, 100 nM A26 RNA target and 2 mM MgCl₂ in Csx1 buffer containing 20 mM 2-(N-morpholino)ethanesulfonic acid (MES) pH 5.5, 100 mM K-glutamate and 1 mM DTT for 2 h at 70° C. in a 100 μl reaction volume. cOA product was extracted by phenol-chloroform (Ambion) extraction followed by chloroform extraction (Sigma-Aldrich), and stored at −20° C.

For single turnover kinetics experiments SIRV1 gp29 and variants (4 μM protein dimer) were assayed for radiolabelled cA₄ degradation by incubating with 1/400 diluted ³²P-labelled SsoCsm cOA (˜80 nM; generated in a 100 μl cOA synthesis reaction) in Csx1 buffer at 50° C. Whereas Bacillus subtilis YddF (8 μM dimer), THSA-485A Tsac_2833 (8 μM dimer) and CRENPOLY SF2_1390015 (2 μM dimer) were incubated with 1/400 diluted ³²P-labelled SsoCsm cOA at 37° C. in K buffer and B buffer described in Table 2. At desired time points, a 10 μl aliquot of the reaction was removed and quenched by adding to chilled phenol-chloroform. Subsequently, 5 μl of deproteinised reaction product was extracted into 5 μl 100% formamide xylene-cyanol loading dye if intended for denaturing polyacrylamide gel electrophoresis (PAGE), or products were further isolated by chloroform extraction if indented for thin-layer chromatography (TLC). A reaction incubating cOA in buffer without protein to the endpoint of each experiment was included as a control. All experiments were carried out in triplicate. cA₄ degradation was visualised by phosphor imaging following denaturing PAGE (7M Urea, 20% acrylamide, 1×TBE) or TLC.

For thin layer chromatography (TLC), 1 μl of radiolabelled product was spotted 1 cm from the bottom of a 20×20 cm silica gel TLC plate (Supelco Sigma-Aldrich). The TLC plate was then placed in a sealed glass chamber pre-warmed at 37° C. and containing 0.5 cm of a running buffer composed of 30% H₂O, 70% ethanol and 0.2 M ammonium bicarbonate, pH 9.2. The buffer was allowed to rise along the plate through capillary action until the migration front reached 15 cm. The plate was then air dried and sample migration was visualised by phosphor imaging.

For kinetic analysis, cA₄ cleavage was quantified using the Bio-Formats plugin (24) of ImageJ as distributed in the Fiji package (25) and fitted to a single exponential curve (y=m1+m2*(1−exp(−m3*x));m1=0.1;m2=1;m3=1;) using Kaleidagraph (Synergy Software), as described previously (Stenberg et al, RNA, 2012, 18(4): 661-672).

The compositions of the reaction buffers used in these methods are shown in Table 2 below.

TABLE 2

| Reaction | | Protein assayed |
|---|---|---|
| buffer name | Composition (1X) | in buffer |
| Csx1 buffer | 20 mM MES pH 5.5, | SIRV1 gp29 (s-t |
| | 100 mM K-glutamate, | kinetics) |
| | 1 mM DTT, 3 units | |
| | SUPERase•In ™ | |
| | Inhibitor | |
| B buffer | 20 mM MES pH 6.0, | SsoCsm, SIRV1 gp29 |
| | 100 mM NaCl, 1 mM | and SsoCsx1 (coupled |
| | DTT, 3 units | deactivation assay) |
| | SUPERase•In ™ | SIRV1 gp29 and |
| | Inhibitor | StCsm6′ (coupled |
| | | deactivation assay) |
| | | THSA-485A Tsac_2833 |
| | | (s-t kinetics) |
| | | THSA-485A Tsac_2833 |
| | | and StCsm6′ (coupled |
| | | deactivation assay) |
| K buffer | 20 mM Tris-HCl pH 8.0, | B. subtilis YddF |
| | 150 mM NaCl, 1 mM | (s-t kinetics) |
| | DTT, 1 mM EDTA, 3 | |
| | units SUPERase•In ™ | |
| | Inhibitor | |

Deactivation of HEPN Nucleases by Ring Nucleases in Coupled Assays

Csm complex (4 μg; μ140 nM Csm carrying crRNA targeting A26) was incubated at 70° C. for 60 min in the presence of Sso2081 (2 μM dimer) or SIRV1 gp29 (2 μM dimer) and A26 RNA target (50, 20, 5, 2, or 0.5 nM) in buffer containing 20 mM MES pH 6.0, 100 mM NaCl, 2 mM MgCl₂ and 0.5 mM ATP. 5′-end labelled A1 RNA (AGGGUAUUAUUUGUUUGUUUCUUCUAAACUAUAAGCUAGUUCUGGAGA) and 0.5 μM dimer SsoCsx1 was then added to the reaction at 60 min and the reaction was allowed to proceed for a further 60 min before quenching by deproteination with phenol-chloroform extraction. A1 RNA cleavage was visualised by phosphor imaging after denaturing PAGE. Control reactions without SIRV1 gp29 were also included to compare to the effect of ring nuclease presence. cA₄ synthesis by SsoCsm in response to A26 RNA target and subsequent cA₄ degradation by Sso2081 or SIRV1 gp29, if present, was visualised by adding 5 nM α-³²P-ATP with the 0.5 mM ATP at the start of the reaction. Reactions were quenched at 60 min and cA₄ degradation products were visualised by phosphor imaging following TLC. Control reactions incubating RNA in buffer, RNA with SsoCsx1 in the absence of Csm, RNA with Csm or SIRV1 gp29 with RNA for 60 min were also carried out.

For evaluation of the cA₄ degradation capacity of the ring nuclease SIRV1 gp29 versus the cellular ring nuclease Sso2081, SIRV1 gp29 (2 μM dimer) was incubated with 200-0.5 μM unlabelled cA₄ (BIOLOG Life Science Institute, Bremen, Germany) in Csx1 buffer at 70° C. for 30 min before introducing SsoCsx1 (0.5 μM dimer) and radio-labelled A1 RNA (50 nM). The reaction was left to proceed for a further 60 min at 70° C. before deproteinising by phenol-chloroform extraction before denaturing PAGE to visualize RNA degradation.

When investigating cA₆ degradation capacity, THSA-485A Tsac_2833 (2 μM dimer) was incubated with cA₆ (50-0.5 μM) for 20 min or 60 min at 37° C. prior to adding 0.5 μM dimer StCsm6′ and 50 nM radio-labelled RNA A1. Reactions were then left to proceed for 60 min at 37° C. before quenching reactions by phenol-chloroform extraction and visualising RNA degradation by phosphor imaging following denaturing PAGE.

Substrate Specificity of Ring Nuclease SIRV Gp29 (Orf 114a)

For determining ring nuclease specificity toward cA4 and cA6, 49 μM SIRV1 gp29 was incubated with 400 μM cA4, cA6, cyclic-AMP, cyclic-di-AMP, cyclic-di-GMP, or cyclic-GAMP at 70° C. for 60 min. The reactions were then deproteinised by phenol-chloroform extraction followed by chloroform extraction and products separated by TLC. Substrate degradation was visualised and imaged under short-wave (254 nM) UV light.

Liquid Chromatography High-Resolution Mass Spectrometry

SIRV1 gp29 (40 μM dimer) was incubated with 400 μM cA₄ in Csx1 buffer for 2 min or 60 min at 70° C. and deproteinised by phenol-chloroform extraction followed by chloroform extraction. Liquid chromatography-high resolution mass spectrometry (LC-HRMS) analysis was performed on a Thermo Scientific Velos Pro instrument equipped with HESI source and Dionex UltiMate 3000 chromatography system. Compounds were separated on a Kinetex 2.6 μm EVO C18 column (2.1×100 mm, Phenomenex) using a linear gradient of acetonitrile (B) against 20 mM ammonium bicarbonate (A): 0-5 min 2% B, 5-33 min 2-15% B, 33-35 min 15-98% B, 35-40 min 98% B, 40-41 min 98-2% B, 41-45 min 2% B. The flow rate was 350 μl min⁻¹ and column temperature was 40° C. UV data were recorded at 254 nm. Mass data were acquired on the FT mass analyser in negative ion mode with scan range m/z 150-1500 at a resolution of 30,000. Source voltage was set to 3.5 kV, capillary temperature was 350° C., and source heater temperature was 250° C. Data were analysed using Xcalibur (Thermo Scientific).

Plasmid Immunity from a Reprogrammed Type III System in E. coli

Plasmids pCsm1-5_ΔCsm6 (containing the type III Csm interference genes cas10, csm3, csm4, csm5 from M. tuberculosis and csm2 from M. canettii), pCRISPR_TetR (containing M. tuberculosis cas6 and tetracycline resistance gene-targeting CRISPR array), pRAT-Target (tetracycline-resistance, target plasmid) and M. tuberculosis (Mtb)Csm6/Thioalkalivibrio sulfidiphilus (Tsu)Csx1 expression constructs were used. pRAT-Duet was constructed by replacing the pUC19 lacZα gene of pRAT-Target with the multiple cloning sites (MCSs) of pACYCDuet-1 by restriction digest (5′-NcoI, 3′-XhoI). The viral ring nuclease (duf1874) gene from Thermoanaerobacterium phage THSA_485A, tsac_2833, was PCR-amplified from its pEHisTEV expression construct and cloned into the 5′-NdeI, 3′-XhoI sites of MCS-2. The cOA-dependent nuclease (tsu csx1) was cloned into the 5′-NcoI, 3′-SalI sites of MCS-1 by restriction digest. Csx1 was cloned with and without the viral ring nuclease; pRAT-Duet without insert and pRAT-Duet containing only the viral ring nuclease were used as controls. E. coli C43 containing pCsm1-5_ΔCsm6 and pCRISPR_TetR were transformed with 100 ng of pRAT-Duet target plasmid containing different combinations of cOA-dependent nuclease and viral ring nuclease. After outgrowth at 37° C. for 2 h, cells were collected and resuspended in 200 μl LB. A series of 10-fold dilutions was applied onto LB agar containing 100 μg ml⁻¹ ampicillin and 50 μg ml⁻¹ spectinomycin to determine the cell density of the recipient cells and onto LB agar additionally containing 25 μg ml⁻¹ tetracycline, 0.2% (w/v) d-lactose and 0.2% (w/v) I-arabinose to determine the cell density of viable transformants. Plates were incubated at 37° C. for 16-18 h; further incubation was carried out at room temperature.

Results

Identification of the Ring Nuclease Family of Anti-CRISPRs

Previously, the inventors investigated a number of hypothetical proteins in archaeal viruses of unknown function. As part of that study, the structures of ORF 114a of the Sulfolobus islandicus rudivirus 1 (SIRV1), ORF 109 from Acidianus filamentous virus 3 (AFV3) and ORF B116 of Sulfolobus turreted icosahedral virus (STIV) were solved and found to be closely related (see, for example, Oke et al. (2010) J Struct Funct Genomics 11:167-180, J Keller et al (2007), Virol J 4:12 and Larson et al (2007) Virology 363(2):387-396(18). In particular, these proteins were found to share a dimeric organisation with a central pocket flanked by conserved residues.

These proteins are part of a widely conserved family with members present in a variety of archaeal virus genomes and baceteriophages (FIG. 1). Furthermore, clear homologues are present in a range of bacterial genomes—for example gene yddF of Bacillus subtilis, which is encoded by a prophage integrated in the genome, and also Desuflurella multipotens, Methylomagnum ischizawi, Crenothrix polyspora and Nitrosomonas marina (also shown in FIG. 1).

The function of this protein family was not known. However, disruption of ORF B116 in STIV resulted in a viable virus with delayed infection kinetics and a marked small plaque phenotype (Wirth et al (2011) Virology 415(1):6-11) and B116 is expressed early in the STIV infection cycle (Ortmann et al (2008) J Virol 82(10):4874-4883).

Therefore the present inventors hypothesised that this family may have an important role in the initiation of infection and explored possibility of an aCR function using SIRV1 gp29 (orf114a) as an exemplar.

It was surprisingly discovered that SIRV1 gp29 has a potent ring nuclease activity. As shown in FIG. 2, SIRV1 gp29 is specific for the degradation of cA₄ and cA₆, and does not recognise the cyclic dinucleotides c-diAMP, c-diGMP and c-CAMP as substrates. In addition, SIRV1 gp29 has been shown to degrade cA₄ with a rate of 7±1 min⁻¹ under single turnover conditions at 50° C. This degradation rate was found to be at least 200× faster than that observed with the cellular ring nuclease Sso2081 (FIG. 3a).

The initial product of this reaction was determined as linear di-adenylate (ApA>P) with a cyclic 2′,3′ phosphate, but this was rapidly converted to ApAp with a 3′ phosphate (FIG. 3b).

Mutation of the conserved histidine H47 to an alanine (H47A variant) reduces the catalytic rate constant by 50,000-fold, indicating the important role of this residue in catalysis (FIG. 3c).

In keeping with the rapid kinetics, SIRV1 gp29 was also observed to de-activate the cA₄-activated HEPN nuclease Csx1 far more effectively than the cRN Sso2081, over a wider range of RNA and cA₄ concentrations (FIG. 4).

Other Ring Nucleases

Three further DUF1874 family members: Yddf from Bacillus subtilis, Tsac_2833 from the bacteriophage THSA-485A and WP_087145848.1 from the bacterium Crenothrix polyspora have been tested for ring nuclease activity.

All three have been shown to be highly active ring nucleases in vitro (FIG. 5). Based on this observation, it is therefore believed that all members of the DUF1874 family with the “GH” active site motif will show ring nuclease activity.

Degradation of cA6

The family of ring nucleases disclosed herein have also been observed to degrade cA₆, albeit with slower kinetics.

A deactivation assay was carried out using the S. thermophilus HEPN ribonuclease StCsm6′, a ribonuclease activated by cA₆. Clear inhibition of substrate RNA cleavage was observed at lower concentrations of cA₆ when the activator was pre-incubated with the ring nuclease Tsac_2833 (FIG. 6). In addition, clear inhibition of substrate RNA cleavage was observed at lower concentrations of cA₆ when the activator was pre-incubated with the vRN SIRV1 gp29 (FIG. 7).

This demonstrates that the ring nuclease family described herein have a broad specificity and can degrade both the cOA activators described in the literature.

Structure and Mechanism of the DUF1874 Ring Nuclease Family

The structures of DUF1874 ring nuclease family members are believed to share a central binding pocket lined by conserved residues that may play a role in catalysis. Prominent amongst these is a histidine residue that may be absolutely conserved across the ring nuclease family.

Starting with the structure of SIRV1 gp29, the cOA molecule cA₄ was modelled into the binding site (FIG. 8). The central binding pocket appears to accommodate the cA₄ molecule snugly, and the conserved histidine is positioned on either side of the binding pocket in a position consistent with a role as a general acid or base in the catalytic cycle.

Since the degradation of cA₄ is believed to be independent of the presence of a metal ion and generates initial products with a cyclic 2′,3′ phosphate, it is hypothesised that the mechanism is related to the cellular ring nucleases whereby the 2′-hydroxyl of the substrate acts as the nucleophile. The role of the conserved histidine in the ring nucleases disclosed herein may be to act as a proton donor and acceptor during catalysis, which could explain the much more rapid cA₄ degradation kinetics compared to the cRNs.

Phylogenetic Distribution and Genomic Context of the DUF1874 Family

Members of the ring nuclease family disclosed herein may be annotated as DUF1874 (domain of unknown function). The gene is most prominent in the archaeal viruses, where it found in at least seven distinct viral classes (FIG. 1). Within the domain Archaea, homologues have been identified in representatives of the Crenarchaeota, Euryarchaeota, Aigarchaeota, Bathyarchaeota and Thorarchaeota. The ring nuclease gene present in S. acidocaldarius is known to be part of an integrated STIV viral genome, and the genome contexts of ring nuclease in archaea show no evidence of association with CRISPR loci, but rather suggest viral origin with adjacent viral-derived orfs.

A clear homologue has been identified in a bacteriophage genome (THSA-485A of the Siphoviridae family, infecting the clostridial species Thermoanaerobacterium saccharolyticum). However, this may reflect the lack of sequence information for phage. In bacterial genomes, orthologues have been found in the Firmicutes including multiple bacilli and clostridia species, cyanobacteria and also in representatives of the alpha, beta, delta and gamma-proteobacteria. The yddF gene in B. subtilis is part of an integrated prophage, but in other species such as Methylomagnum ishizawai, Crenothrix polyspora, Methylovulum psychrotolerans and Nitrosomonas marina the ring nuclease gene is associated with type III CRISPR systems. There is also an example (Marinitoga piezophilia uniprot accession H2J4R5) of DUF1874 fused to a cOA-activated HEPN ribonuclease of the Csx1 family. Since both active sites are conserved, this fusion protein may have cA₄ activated ribonuclease activity coupled with a cA₄ degradative ring nuclease. This fusion protein may therefore provide an explicit linkage between the DUF1874 family and the type III CRISPR system.

Use of a DUF1874 Ring Nuclease in Providing Type III CRISPR Immunity In Vivo

A recombinant type III CRISPR system from Mycobacterium tuberculosis in an Escherichia coli host was used to explore efficacy of ring nucleases in vivo. A strain that was capable of cA₄-based immunity was transformed with a plasmid that was targeted for interference due to a match in the tetracycline resistance gene to a spacer in the CRISPR array. Efficient interference (lack of plasmid transformation) was observed after one day in the absence of the duf1874 gene (Tsac_2833) from bacteriophage THSA-485A. However, the presence of the duf1874 gene (Tsac_2833 THSA-486A) on the plasmid reduced immunity for cA₄-mediated CRISPR defence. These results are shown in FIG. 9.

These observations indicate that a DUF1874 can act as a ring nuclease against cA₄-mediated type III CRISPR defence.

### REFERENCES

1. Pursey E, Sunderhauf D, Gaze W H, Westra E R, & van Houte S (2018) CRISPR-Cas antimicrobials: Challenges and future prospects. PLoS Pathog 14(6):e1006990.
2. Borges A L, Davidson A R, & Bondy-Denomy J (2017) The Discovery, Mechanisms, and Evolutionary Impact of Anti-CRISPRs. Annu Rev Virol 4(1):37-59.
3. Pyenson N C, Gayvert K, Varble A, Elemento O, & Marraffini L A (2017) Broad Targeting Specificity during Bacterial Type III CRISPR-Cas Immunity Constrains Viral Escape. Cell Host Microbe 22(3):343-353e343.
4. Kazlauskiene M, Kostiuk G, Venclovas C, Tamulaitis G, & Siksnys V (2017) A cyclic oligonucleotide signaling pathway in type III CRISPR-Cas systems. Science 357(6351):605-609.
5. Niewoehner O, Garcia-Doval C, Rostol J T, Berk C, Schwede F, Bigler L, Hall J, Marraffini L A, & Jinek M (2017) Type III CRISPR-Cas systems produce cyclic oligoadenylate second messengers. Nature 548(7669):543-548.
6. Rouillon C, Athukoralage J S, Graham S, Grüschow S, & White M F (2018) Control of cyclic oligoadenylate synthesis in a type III CRISPR system. eLife 7:e36734.
7. Makarova K S, Anantharaman V, Grishin N V, Koonin E V, & Aravind L (2014) CARF and WYL domains: ligand-binding regulators of prokaryotic defense systems. Frontiers in genetics 5:102.
8. Sheppard N F, Glover C V, 3rd, Terns R M, & Terns M P (2016) The CRISPR-associated Csx1 protein of Pyrococcus furiosus is an adenosine-specific endoribonuclease. RNA 22(2):216-224.
9. Deng L, Garrett R A, Shah S A, Peng X, & She Q (2013) A novel interference mechanism by a type IIIB CRISPR-Cmr module in Sulfolobus. Mol. Microbiol. 87(5):1088-1099.
10. Jiang W, Samai P, & Marraffini L A (2016) Degradation of Phage Transcripts by

CRISPR-Associated RNases Enables Type III CRISPR-Cas Immunity. Cell 164(4):710-721.
11. Foster K, Kalter J, Woodside W, Terns R M, & Terns M P (2018) The ribonuclease activity of Csm6 is required for anti-plasmid immunity by Type III-A CRISPR-Cas systems. RNA Biol:1-12.
12. Athukoralage J S, Rouillon C, Graham S, Grüschow S, & White M F (2018) Ring nucleases deactivate Type III CRISPR ribonucleases by degrading cyclic oligoadenylate. Nature 562(7726):277-280.
13. Pawluk A, Steals R H, Taylor C, Watson B N, Saha S, Fineran P C, Maxwell K L, & Davidson A R (2016) Inactivation of CRISPR-Cas systems by anti-CRISPR proteins in diverse bacterial species. Nat Microbiol 1(8):16085.
14. Maxwell K L (2016) Phages Fight Back: Inactivation of the CRISPR-Cas Bacterial Immune System by Anti-CRISPR Proteins. PLoS Pathog 12(1):e1005282.
15. He F, Bhoobalan-Chitty Y, Van L B, Kjeldsen A L, Dedola M, Makarova K S, Koonin E V, Brodersen D E, & Peng X (2018) Publisher Correction: Anti-CRISPR proteins encoded by archaeal lytic viruses inhibit subtype I-D immunity. Nat Microbiol 3(9):1076.
16. Oke M, Carter L G, Johnson K A, Liu H, McMahon S A, Yan X, Kerou M, Weikart N D, Kadi N, Sheikh M A, et al. (2010) The Scottish Structural Proteomics Facility: targets, methods and outputs. J Struct Funct Genomics 11:167-180.
17. Keller J, Leulliot N, Cambillau C, Campanacci V, Porciero S, Prangishvili D, Forterre P, Cortez D, Quevillon-Cheruel S, & van Tilbeurgh H (2007) Crystal structure of AFV3-109, a highly conserved protein from crenarchaeal viruses. Virol J 4:12.
18. Larson E T, Eilers B J, Reiter D, Ortmann A C, Young M J, & Lawrence C M (2007) A new DNA binding protein highly conserved in diverse crenarchaeal viruses. Virology 363(2):387-396.
19. Wirth J F, Snyder J C, Hochstein R A, Ortmann A C, Willits D A, Douglas T, & Young M J (2011) Development of a genetic system for the archaeal virus Sulfolobus turreted icosahedral virus (STIV). Virology 415(1):6-11.
20. Ortmann A C, Brumfield S K, Walther J, Mclnnerney K, Brouns S J, van de Werken H J, Bothner B, Douglas T, van de Oost J, & Young M J (2008) Transcriptome analysis of infection of the archaeon Sulfolobus solfataricus with Sulfolobus turreted icosahedral virus. J Virol 82(10):4874-4883.
21. Rouillon C, Athukoralage J S, Graham S, Grüschow S, & White M F (2019) Investigation of the cyclic oligoadenylate signalling pathway of type III CRISPR systems. Method Enzymol 616:191-218.
22. Yang W (2011) Nucleases: diversity of structure, function and mechanism. Q Rev Biophys 44(1):1-93.
23. Anderson R E, Kouris A, Seward C H, Campbell K M, & Whitaker R J (2017) Structured

Populations of Sulfolobus acidocaldarius with Susceptibility to Mobile Genetic Elements. Genome biology and evolution 9(6):1699-1710.
24. Kelley L A & Sternberg M J (2009) Protein structure prediction on the Web: a case study using the Phyre server. Nature protocols 4(3):363-371.
25. Sulakvelidze A, Alavidze Z, & Morris J G, Jr. (2001) Bacteriophage therapy. Antimicrob Agents Chemother 45(3):649-659.
26. Louwen R, Staals R H, Endtz H P, van Baarlen P, & van der Oost J (2014) The role of CRISPR-Cas systems in virulence of pathogenic bacteria. Microbiol Mol Biol Rev 78(1):74-88.
27. Gootenberg J S, Abudayyeh 00, Kellner M J, Joung J, Collins J J, & Zhang F (2018) Multiplexed and portable nucleic acid detection platform with Cas13, Cas12a, and Csm6. Science 360(6387):439-444.
28. Linkert M, Rueden C T, Allan C, Burel J M, Moore W, Patterson A, Loranger B, Moore J, Neves C, Macdonald D, et al. (2010) Metadata matters: access to image data in the real world. J Cell Biol 189(5):777-782.
29. Schindelin J, Arganda-Carreras I, Frise E, Kaynig V, Longair M, Pietzsch T, Preibisch S, Rueden C, Saalfeld S, Schmid B, et al. (2012) Fiji: an open-source platform for biological-image analysis. Nat Methods 9(7):676-682.
30. Sternberg S H, Haurwitz R E, & Doudna J A (2012) Mechanism of substrate selection by a highly specific CRISPR endoribonuclease. RNA 18(4):661-672.

1. A method of modulating the function, structure and/or activity of cyclic oligoadenylate (cOA), said method comprising contacting the cOA with a DUF1874 protein, wherein the DUF1874 protein acts as a ring nuclease enzyme.

2. The method of claim 1, wherein the DUF1874 protein comprises one or more of the features selected from the group consisting of:
(i) a conserved catalytic site for the degradation of cyclic oligoadenylate (cOA);
(ii) a key catalytic histidine residue; and
(iii) a GH active site motif.

3. The method of claim 1, wherein the DUF1874 protein comprises a sequence selected from the group consisting of:
(i) SEQ ID NO 1;
(ii) SEQ ID NO: 2;
(iii) SEQ ID NO: 3;
(iv) SEQ ID NO: 4; and
(v) SEQ ID NO: 5.

4. The method of claim 1, wherein the DUF1874 protein comprises a sequence at least 95% identical to one or more sequences selected from the group consisting of:
(i) SEQ ID NO: 6;
(ii) SEQ ID NO: 7;
(iii) SEQ ID NO: 8;
(iv) SEQ ID NO: 9;
(v) SEQ ID NO: 10;
(vi) SEQ ID NO: 11;
(vii) SEQ ID NO: 12;
(viii) SEQ ID NO: 13;
(ix) SEQ ID NO: 14;
(x) SEQ ID NO: 15;
(xi) SEQ ID NO: 16;
(xii) SEQ ID NO: 17;
(xiii) SEQ ID NO: 18; and
(xiv) A functional fragment of any one of sequences (i)-(xiii).

5. The method of claim 1, wherein the DUF1874 protein comprises a sequence at least 95% identical to SEQ ID NO: 6 (THSA-485A (orf Tsac_2833)).

6.-7. (canceled)

8. A method of treating and/or preventing a bacterial infection or disease or condition associated with a bacterial infection, said method comprising administering a subject in need thereof a DUF1874 protein.

9. The method of claim 8, wherein the bacterial infection is caused or contributed to by one or more bacteria selected from the group consisting of:
(i) Mycobacterium tuberculosis
(ii) Neisseria meningitidis
(iii) Neisseria mucosa
(iv) Staphylococcus aureus
(v) Streptococcus pyogenes
(vi) Streptococcus mutans
(vii) Pectobacterium carotovorum; and
(viii) Dickeya dadantii;
and the disease or condition is selected from the group consisting of:
(i) tuberculosis;
(ii) bacterial meningitis and sepsis;
(iii) endocarditis;
(iv) sepsis and toxic shock;
(v) sepsis, pharyngitis;
(vi) tooth decay;
(vii) ubiquitous plant pathogen; and
(xiii) crop pathogen.

10.-13. (canceled)

14. A method of detecting a ring nuclease amino acid or nucleic acid sequence in a sample, said method comprising probing said sample for the presence of a sequence corresponding to any one of SEQ ID NOS: 1-24, a nucleic acid encoding any one of SEQ ID NOS: 1-24 or a function fragment of any of these sequences, wherein detection of a sequence corresponding to any one of SEQ ID NOS: 1-24, a nucleic acid encoding any one of SEQ ID NOS: 1-24 or a function fragment of any of these sequences indicates that the sample comprises a ring nuclease amino acid or nucleic acid sequence.

15. A method of identifying a modulator of a ring nuclease having a sequence at least 95% identical to a sequence provided by any one of SEQ ID NOS: 1-24 or a functional fragment thereof, said method comprising:
contacting a test agent with a ring nuclease having a sequence at least 95% identical to a sequence provided by any one of SEQ ID NOS: 1-24 or a functional fragment thereof;
assessing the function or activity of the ring nuclease enzyme in the presence of the test agent;
wherein modulation of the function or activity of the ring nuclease indicates that the test agent may be a ring nuclease modulator.

16. The method of claim 15, wherein the function or activity of the ring nuclease enzyme may be compared to a standard or normal level of ring nuclease activity or function.

17.-18. (canceled)


SEQUENCE LISTING (25 sequences)

SEQ ID NO: 1 - protein, 132 aa, Artificial sequence
  Synthetic consensus sequence
  misc_feature (1)..(1): Xaa can be any naturally occurring amino acid
  MISC_FEATURE (2)..(50): Xaa is present or absent and when present denotes any amino acid.
  MISC_FEATURE (53)..(132): Xaa is present or absent and when present denotes any amino acid.
XXXXXXXXXXXXXXXXXXXXXXXXXXXXXXXXXXXXXXXXXXXXXXXXXXGHXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 2 - protein, 177 aa, Artificial Sequence
  Synthetic consensus sequence
  misc_feature (1)..(1): Xaa can be any naturally occurring amino acid
  MISC_FEATURE (2)..(10): Xaa is present or absent and when present denotes any amino acid.
  misc_feature (12)..(30): Xaa can be any naturally occurring amino acid
  MISC_FEATURE (31)..(61): Xaa is present or absent and when present denotes any amino acid.
  misc_feature (64)..(73): Xaa can be any naturally occurring amino acid
  MISC_FEATURE (74)..(88): Xaa is present or absent and when present denotes any amino acid.
  misc_feature (91)..(100): Xaa can be any naturally occurring amino acid
  MISC_FEATURE (101)..(115): Xaa is present or absent and when present denotes any amino acid.
  MISC_FEATURE (117)..(126): Xaa is present or absent and when present denotes any amino acid.
  misc_feature (128)..(132): Xaa can be any naturally occurring amino acid
  MISC_FEATURE (133)..(177): Xaa is present or absent and when present denotes any amino acid.
XXXXXXXXXXNXXXXXXXXXXXXXXXXXXXXXXXXXXXXXXXXXXXXXXXXXXXXXXXXXXGHXXXXXXXXXXXXXXXXXXXXXXXXXNRXXXXXXXXXXXXXXXXXXXXXXXXXRXXXXXXXXXXEXXXXXXXXXXXXXXXXXXXXXXXXXXXXXXXXXXXXXXXXXXXXXXXXXX

SEQ ID NO: 3 - protein, 195 aa, Artificial Sequence
  Synthetic consensus sequence
  misc_feature (1)..(1): Xaa can be any naturally occurring amino acid
  MISC_FEATURE (2)..(10): Xaa is present or absent and when present denotes any amino acid.
  misc_feature (12)..(36): Xaa can be any naturally occurring amino acid
  MISC_FEATURE (37)..(61): Xaa is present or absent and when present denotes any amino acid.
  misc_feature (64)..(64): Xaa can be any naturally occurring amino acid
  MISC_FEATURE (65)..(73): Xaa is present or absent and when present denotes any amino acid.
  MISC_FEATURE (74)..(74): Xaa denotes Thr, Ala, Ser or Gly.
  misc_feature (75)..(75): Xaa can be any naturally occurring amino acid
  MISC_FEATURE (76)..(94): Xaa is present or absent and when present denotes any amino acid.
  misc_feature (97)..(101): Xaa can be any naturally occurring amino acid
  MISC_FEATURE (102)..(111): Xaa is present or absent and when present denotes any amino acid.
  MISC_FEATURE (112)..(112): Xaa denotes Asp or Gln.
  misc_feature (113)..(116): Xaa can be any naturally occurring amino acid
  MISC_FEATURE (117)..(132): Xaa is present or absent and when present denotes any amino acid.
  MISC_FEATURE (134)..(143): Xaa is present or absent and when present denotes any amino acid.
  MISC_FEATURE (145)..(145): Xaa denotes Gly or Asn.
  misc_feature (146)..(150): Xaa can be any naturally occurring amino acid
  MISC_FEATURE (151)..(195): Xaa is present or absent and when present denotes any amino acid.
XXXXXXXXXXNXXXXXXXXXXXXXXXXXXXXXXXXXXXXXXXXXXXXXXXXXXXXXXXXXXGHXXXXXXXXXXXXXXXXXXXXXXXXXXXXXXXNRXXXXXXXXXXXXXXXXXXXXXXXXXXXXXXXXXXXXRXXXXXXXXXXEXXXXXXXXXXXXXXXXXXXXXXXXXXXXXXXXXXXXXXXXXXXXXXXXXXX

SEQ ID NO: 4 - protein, 200 aa, Artificial Sequence
  Synthetic consensus sequence
  misc_feature (1)..(1): Xaa can be any naturally occurring amino acid
  MISC_FEATURE (2)..(10): Xaa is present or absent and when present denotes any amino acid.
  misc_feature (12)..(35): Xaa can be any naturally occurring amino acid
  MISC_FEATURE (36)..(61): Xaa is present or absent and when present denotes any amino acid.
  MISC_FEATURE (64)..(73): Xaa is present or absent and when present denotes any amino acid.
  MISC_FEATURE (75)..(94): Xaa is present or absent and when present denotes any amino acid.
  MISC_FEATURE (97)..(116): Xaa is present or absent and when present denotes any amino acid.
  MISC_FEATURE (118)..(137): Xaa is present or absent and when present denotes any amino acid.
  MISC_FEATURE (139)..(148): Xaa is present or absent and when present denotes any amino acid.
  misc_feature (151)..(160): Xaa can be any naturally occurring amino acid
  MISC_FEATURE (161)..(200): Xaa is present or absent and when present denotes any amino acid.
XXXXXXXXXXNXXXXXXXXXXXXXXXXXXXXXXXXXXXXXXXXXXXXXXXXXXXXXXXXXXGHXXXXXXXXXXTXXXXXXXXXXXXXXXXXXXXNRXXXXXXXXXXXXXXXXXXXXDXXXXXXXXXXXXXXXXXXXXRXXXXXXXXXXEGXXXXXXXXXXXXXXXXXXXXXXXXXXXXXXXXXXXXXXXXXXXXXXXXXX

SEQ ID NO: 5 - protein, 200 aa, Artificial Sequence
  Synthetic consensus sequence
  misc_feature (1)..(1): Xaa can be any naturally occurring amino acid
  MISC_FEATURE (2)..(10): Xaa is present or absent and when present denotes any amino acid.
  misc_feature (12)..(35): Xaa can be any naturally occurring amino acid
  MISC_FEATURE (36)..(61): Xaa is present or absent and when present denotes any amino acid.
  MISC_FEATURE (64)..(73): Xaa is present or absent and when present denotes any amino acid.
  MISC_FEATURE (75)..(94): Xaa is present or absent and when present denotes any amino acid.
  MISC_FEATURE (97)..(116): Xaa is present or absent and when present denotes any amino acid.
  MISC_FEATURE (118)..(137): Xaa is present or absent and when present denotes any amino acid.
  MISC_FEATURE (139)..(148): Xaa is present or absent and when present denotes any amino acid.
  misc_feature (151)..(160): Xaa can be any naturally occurring amino acid
  MISC_FEATURE (161)..(200): Xaa is present or absent and when present denotes any amino acid.
XXXXXXXXXXNXXXXXXXXXXXXXXXXXXXXXXXXXXXXXXXXXXXXXXXXXXXXXXXXXXGHXXXXXXXXXXSXXXXXXXXXXXXXXXXXXXXNRXXXXXXXXXXXXXXXXXXXXDXXXXXXXXXXXXXXXXXXXXRXXXXXXXXXXEGXXXXXXXXXXXXXXXXXXXXXXXXXXXXXXXXXXXXXXXXXXXXXXXXXX

SEQ ID NO: 6 - protein, 102 aa, Archael virus
MFIANAFSLQMLSQFPAHIDIEEVATSAVAKLDLQSAIGHADTAVVLSGILGKDIESNRVNVQLQPGDSLIVAQLMGGRLPEGSTTLPAGFSFKFFKVTVQA

SEQ ID NO: 7 - protein, 114 aa, Archael virus
MNKVYLANAFSINMLTKFPTKVVIDKIDRLEFCENIDNEDIINSIGHDSTIQLINSLCGTTFQKNRVEIKLEKEDKLYVVQISQRLEEGKILTLEEILKLYESGKVQFFEIIVD

SEQ ID NO: 8 - protein, 116 aa, Archael virus
MGKVFLTNAFSINMLKEFPTTITIDKLDEEDFCLKLELRLEDGTLINAIGHDSTINLVNTLCGTQLQKNRVEVKMNEGDEALIIMISQRLEEGKVLSDKEIKDMYRQGKISFYEVW

SEQ ID NO: 9 - protein, 109 aa, Archael virus
MLYILNSAILPLKPGEEYTVKAKEITIQEAKELVTKEQFTSAIGHQATAELLSSILGVNVPMNRVQIKVTHGDRILAFMLKQRLPEGVVVKTTEELEKIGYELWLFEIQ

SEQ ID NO: 10 - protein, 108 aa, Archael virus
MLYILNAQITPFEGAQATFVERRIDVNEAKKIVNSQPFVSAVGHAATAQLLSKLLDASIPTNRTQVFLKPGDMALAIVLKSRIPEGVVLDEQAIRNIGFEIVVIERVS

SEQ ID NO: 11 - protein, 118 aa, Archael virus
MLYILNSATLPLKPGKEYVIHAKELTIEEAKELLENERFISAVGHEATAKMLTNIFDVEIPMNRIQIFLDDGDKLLSIILKTRLEEGKVIKTVEELEQIGYNIWLFEVVTYEHNVKYE

SEQ ID NO: 12 - protein, 113 aa, Archael virus
MTVYLANAFSPSMLNKLPSAVEFQRVDQKEFCEAIHHGVSNAIGHKGTIEFVNTLCNTNLQTNRVEIKAGINDVIYIIVLGFRLEEGKVLSAGEVQKAYDEGKVLLLKAIIGK

SEQ ID NO: 13 - protein, 118 aa, Archael virus
MLYILNSATLPLKPGKEYVIHAKELTIEEAKELLENERFISAVGHEATAKMLTNIFGVEISMNRIQIFLDDGDKLLSIILKTRLEEGKVIKTVEELEQIGYNIWLFEVVTYVHNAKYE

SEQ ID NO: 14 - protein, 116 aa, Archael virus
MGSYLLNGFSPAMLASGHSVVFFNQIPDVMLCGALTSDELVNAIGHKSTINLINRICQTNLKENRIQVMLQDGDEAFIVVVTERLEEGKVLSDEEITKMFEDGKIKIYYARVHSVV

SEQ ID NO: 15 - protein, 108 aa, Archael virus
MEIAFLNSLVVTSPGFYKAEKITLDEVKQWLKHYDGRYKSFIGHKSTAQFLQKLLGIRIEQNRKTFRHMKYQKAICFSLYERYPENVLLTQRDLEKARYQFYLLTRLD

SEQ ID NO: 16 - protein, 106 aa, Crenothrix polyspora
MTLFIINAPILTSYGDWRFEGPLSIDKTRKLLREGFTSAIGHAASAEMLARLLAMDIPVNRIAITMEAGDRALILRLLQRLPEGKVLNHHEMMATPFELALLTKLK

SEQ ID NO: 17 - protein, 105 aa, Nitrosomonas marina
MLYLINSPILTSYGDWQFSGPLTVAEAKSRLSNDFISAIGHQSGAAFLSALLDIEIPVNRIEINMKPGDSALVLRLKSRLPEGKVLTHDEMQQIPYELGWLVRVQ

SEQ ID NO: 18 - protein, 107 aa, Methylomagnum ischizawi
MALIYVINSPVLTGYGLWRFEGPLAESAARELLAGGFVSALGHAGAARFLSARLGLGIPVNRVRVELQPGDRALVLRLLERLPENRALSAAEMEELPFELGLLTRLE

SEQ ID NO: 19 - protein, 105 aa, Desulfurella multipotens
MVYLLNGPILTDFGLYRYKKISILQAKKILKENKFVSAIGHEATAIFLSELLELDIKYNRIAIKMKQGDLAIVFHLLTRLKEGQVLNIKELCSKDYTLGILKRLE

SEQ ID NO: 20 - protein, 117 aa, Archael virus
MGVWSVVLYLLNTLIVPFRDERAKFEIERVSAEEAKKIIQMHNSQFVSAIGHSASANALSLLLGVAVPVNRTEVFFNVGDEAIAMALKKRLAEGQVLRTVQELEAVGFDLYYIKRVQ

SEQ ID NO: 21 - protein, 111 aa, Synechococcus phage S-CBWM1
MACCVVPKGAPGLWSVVEISLEEVIQDLEEGEFISTIGHPSSAHILETLTGFPFEACRREADPRPGDEFYCFILNSRAPEGKILDEHEIYKIGFSFRKMTYVLGKIPTAPD

SEQ ID NO: 22 - protein, 107 aa, Fusobacterium phage Fnu1
MTIGILNTPILTGEGTYKLSNITLEQAQKLVNENEFISYIGHQATAEIISILLGTEVPMNRGQFKQEVGQKAIIFKLKSRLLEGQILLTIQEIEEIGYEFQLLERKN

SEQ ID NO: 23 - protein, 110 aa, Hydrogenobaculum phage 1
MLYVLNSLIVPVDFQNKQGYIVSLWKIDLETARKIVREMPFTSAVGHEATAKVLSELLGVEISFNRITVKMKEGDAGLHFVLRTRLPEGKVLSEEELRQLDFDLVLSRVS

SEQ ID NO: 24 - protein, 104 aa, Archael Virus
MEIAFLNSLVVTSPGFYKAEKITLDELKHYDGRYKSFIGHKSTAQFLQKLLGIRIEQNRKTFRHMKYQKAICFSLYERYPENVLLTQRDLEKARYQFYLLTRLD

SEQ ID NO: 25 - RNA, 48 nt, Artificial Sequence
  Target RNA
aggguauuauuuguuuguuucuucuaaacuauaagcuaguucuggaga